SEQUENCE LISTING (30 sequences)

SEQ ID NO: 1 - DNA, 41 bp, Artificial Sequence
  Synthetic
gaaggtgaccaagttcatgctgaagaacctgcacccgtcct

SEQ ID NO: 2 - DNA, 41 bp, Artificial Sequence
  Synthetic
gaaggtcggagtcaacggattgaagaacctgcacccgtcac

SEQ ID NO: 3 - DNA, 25 bp, Artificial Sequence
  Synthetic
gcatggaaagaaactgaacaaagat

SEQ ID NO: 4 - DNA, 42 bp, Artificial Sequence
  Synthetic
gaaggtgaccaagttcatgctcaggcaccatgagttatccct

SEQ ID NO: 5 - DNA, 42 bp, Artificial Sequence
  Synthetic
gaaggtcggagtcaacggattcaggcaccatgagttatcccc

SEQ ID NO: 6 - DNA, 28 bp, Artificial Sequence
  Synthetic
tgttatcagtaaatggagagacaaagac

SEQ ID NO: 7 - DNA, 41 bp, Artificial Sequence
  Synthetic
gaaggtgaccaagttcatgctgcaagctcctgaagtccgaa

SEQ ID NO: 8 - DNA, 40 bp, Artificial Sequence
  Synthetic
gaaggtcggagtcaacggattcaagctcctgaagtccgcg

SEQ ID NO: 9 - DNA, 23 bp, Artificial Sequence
  Synthetic
ttctgctgctcttccatctttca

SEQ ID NO: 10 - DNA, 45 bp, Artificial Sequence
  Synthetic
gaaggtgaccaagttcatgctgaagcctcttgatgaagcagcatg

SEQ ID NO: 11 - DNA, 45 bp, Artificial Sequence
  Synthetic
gaaggtcggagtcaacggattgaagcctcttgatgaagcagcata

SEQ ID NO: 12 - DNA, 24 bp, Artificial Sequence
  Synthetic
caacatcgtcctgacaaatggaat

SEQ ID NO: 13 - DNA, 45 bp, Artificial Sequence
  Synthetic
gaaggtgaccaagttcatgcttcctagcactatataaaccccctc

SEQ ID NO: 14 - DNA, 45 bp, Artificial Sequence
  Synthetic
gaaggtcggagtcaacggatttcctagcactatataaacccccta

SEQ ID NO: 15 - DNA, 24 bp, Artificial Sequence
  Synthetic
cttgagtttgctttgcttgaaggc

SEQ ID NO: 16 - DNA, 47 bp, Artificial Sequence
  Synthetic
gaaggtgaccaagttcatgctggctcagtgtttatatagttggagac

SEQ ID NO: 17 - DNA, 46 bp, Artificial Sequence
  Synthetic
gaaggtcggagtcaacggattggctcagtgtttatatagttggaga

SEQ ID NO: 18 - DNA, 27 bp, Artificial Sequence
  Synthetic
gaaaaaaaagcaaaggttagatatgca

SEQ ID NO: 19 - DNA, 7221 bp, Oryza sativa L.
aggacgttggctctgccctgaattatgataaatcatatcgtacatataaattacaaggatacatgagccacctcccctcaaatgcttatatattaattgcacacccttgtcgatcgatcgatctgcgagcgatctggaccgcacaaatataaacgaacggctccgacgcacgcacgtggcatccatagaggatgcacatatacgtacgtaagtacgtatattcacaatgtacgcgtacatacgtacgtatacatacgctataccatacgtatgcatgtcgatcttcttcttctctaccttgggagcgggatgtcggccaggtcgtcgcggtacggcaagggctcgtcgtcgtcgacggcgaggtcgccggcgtcggccttctcggcgtcgacgacggcggcgagggaggagtcggcgacgaaggtgacgacggagtccttcctgatggtgaggtagacgacggcgacgatgtagacgagcatgaacgggaagacggcggcgccgacgagcacgttggcgtacttggggaggtcgttgtggatgagccagccgacgaagctcgtgctcaggaagtacatgttgatgccgatgatgagcagccccaggaaccacgagaacactattatctgcattcaattcaattcaatttattttttgacatacatgacgctaatttattcattgatagcgtcactaaataaccagtcatctttgacgggtggtaacttatatatctttttaacgggtatcgactgatgagcatgcgcaatttttataggtagtatattaatatatatatatatagccatgtgtgtaataataagaaataaatttacatttgctcaattttgttgtagatcagtttatttatggtccttaaaaaagaattacattttctaatgttgacttgtatgtgtctagaaatgtttaagtagagatacaacgatatacgtaattatctatgttaaattatatttacattttacttccatgcaagaaaaaatatatatagtgtactgaagaacttggctcttcgatgtctgtaaagatcaagcaagtacgttactgcatgattttgacacatcatgccgacgacgcaatacatgattaatctctggtactactgcaagacagaaaaatgatgcaggacgtacatcttggtcgtcgttttcatatgtacgacgatcctctatatgcatggctggaccatatatatacctgtcctgcattggcccctgctgtgctatattactattgcatgagctaggtagctaatgattcaattcaattcactgcacatctcgatctacattatgcacacactgacctatagaacatggtgtcctgcaggggttgcatgcatgcagggtgaaggaccagctagctagccaactatcatcattaatttatattctttaccaacaattattgaaactctaatttcatgcatggacagatagatgcatatatttatgttacttgtacatttgctgccatttgagattaagaaacaaactataattaagcaatgcatatatatagtgtacgatcttacatagatagagttcttgtggggccccatcttgctcttactgctgctgaacttgagaagagggatgagagcaaacggcagctcgaaggacagtatcatctgcatgcacacaacacgtacggattaggaacaaattaatcatatatatgtaattaagcaaacaagaactagcaatgatcagatcactactttgatttgatttaattatatataattagtccttacttattgagttggaagctagtctagctagatggtacactgtattatatatatgcatggtgctattttagatcagattgcaaaaaaagaaaagatacctagctagtcagtgacattttgctaatcagatcactactttgatttggcaataatctaaaaaaagaagaaaaagtgcttttgctctaatgacatatatatatgctcttagtactatgtcactaggtacctagtggataccccactactacctagctagctacataaatccgcacgatcgactgcacacacgtgactgtttgggcatgtgcaaaattaaaaatttaaacgcattaaattttaatcgatcggcgacacgacggccggccggttgctcgccggcgggcgccgccgtatggcgcacgcagtttgatatagtaaaatagtaataatagtaaagatttgctatagtaaatagtaataataaaaatgctgaccgaagcgatgatgatgaggcggccggcgcccctggagccgccgatgatggagacgatgaggctcggcgcgatggcgatggttcttgtcatcaggttccgaagccacttcctcatcctgatgtccaagaaaccctgcacatacatggatcatatcatatatatcaacgcgacatgatgatgatttatcatatactccctccgtcattaaatatttgacaccattgatttttctaaacatatttaaccattcatcttattcaataaatttaagtagttattaattcttttcttattatttaattcattgttgtatatactactactactcaagttttttaaataatattcacaaaagtttttgcataagacgaacggtcaaacatatttataaaaaatcaacgacgtcaaatatttagagacggagtgagtatatcctgaagatcagtcctttggagtgaaaaaataaatatatattttcgggtccgatttatcccgtaactgaaatacacttcatatcataaatagattattaagaggaatgagactcgaactagatcggttagcccgccaggtagattaggaaaactagttgttctctttctcaacgttcacttcagaaagaaaattttacggtgcgatgggatatcctattttttttaatataatttatagagtttcgttagcatatttttattaatcaattggttttctaagtatatgtaaacagtaaaatcgctggttattgatgagactgcaccgtgaaatggactagaccaaaagagcggagaaatatggacgaaagtggtaagtagtggcagcaaggtagcgtcttaattcgatcttactttaacaaaaacatatggtgaaatccgacgaggatcgccagattacctaattaataaaggaatattatatcactaaatattcgtgatgtaaacaggattaggtgtgcacccctacaattcgtcagttaacctccataattgaacggattaacaaattaattatgtggcagctagccgtttaattaattatagttcaatcatttaattacgcgctaagctagctggtgatgtacaggtacaggtacagttaagttaattaatttaattaattacctgcatgatgtactgtccagcgtatgtgccggtaatagtggagctctgcccagatgccaacagtgccacgccgtacacgatcgcactcgacttgcccagcacgttctgaaccacataaaaaattgacgtcacaatttgaccataaattagttactcctcctattcaaaaatataaacatatctaataaagttattatattttaggacggagaaatcgtgtagactaattcggtgttattgtggttaattaaatattgattaaattgaacacatacaagctagtactacgaatctgaataaattcctatatctgtagtattaggatacgtctaatccatttaaaaacttttaatattaaagagtaggaagtaataatgaatgaatgaccttgagaaggaaggaggaggtgtcgaggctgaggttggcgcacttgtcggcgtcctcttgggagaggttggcggaggagcaggcggtgccggagacggagacgacggcgatgtttatcagcagcgccacgaacagcgcgaacccgctctcgtacaggaagaacctgcacccgtcaccatatagtacatattaattattagcctggatctttgttcagtttctttccatgcatgaaaaaattcttagctgaatccagtggtactggagcccgaaattatagaagattagtgctaaaataaaacttagctaataaagtattggttttctatgaaatttacatgttaatttaataaaatttaaacaagatttaagagacaggactagatcgagttcatgtcctagctaccctatacctatctcgatctgcccaggctgaagatagctaccttgattcctctgactgatgccggtgtcttcctcgatagcaccaaggcagaatgcaagaacagattgtggctgcatatatataaatgcatgcagtagttaattaactgattagttgtatgaattaattgattgattgttgcgtatgtatgtatgtatgtatgtaaaagcgtacggcatgacaagagctccgaggagggcaatggcgtcggcggtggcgccgtcgccgttgagcctggggatgaagagccccttcatcacctccttcgccggcggcttcacgatgctcagctccccgaagaagcacgccgccatcacgaacaccagcatcgatatcagaaactccagcttcctcacctgttttttttcaaaaaaaattcatcaatcaattttcatgcagttccacagtcagagaaaaaaaaactgaataatgtacatataatatgccatttgagtacttgacaatcgatccaactagctaatttcatatactattccatttttataatcgtttctttgagtgcatcctacgctaatgcttgtgatactaaacaaaaacaacttacaaaaaataatttataccccgtatttttggaggccaagaagcagtagagtgctggtgccggtgatgaggacgccgacccacaccggaatatggaacaatatgttgaaagcaaaggccgtccctataactgcatccaccaaaattaacaacatcaaacgacatgtaattaattaattaattataatcacatcgatctgtacctttctatctgttgatcgatctatctatctatctatatatcacctttgctatcatcatgtgaggtatcttccataactagtttaatttttttaaaaaagaacttgcatgcatgatcaattaaaaaggcaatttacaacggttgaggttaactaatcccgatcagcaaagcttcgcgcagctccgttttgcaactgctacaccactgatctatcttccatgttgctagatagatagatatttagatattgttgttattgtttaaacacacatggatgtatacaggacaggacaaatgtgaaatgacaagagggtgacaaaatagttgtttactaatctgcgtaagatgatgaacgtactcacccagtgtttaaaatctaatcttgatgtgcatcattgacaattttatgggaagctactgtggacgtggtcactactctaaacagctaaaacgcgcatttgcttatgtttgaaacagattattcaacagatatattattggtacatattatctaccttattatatggatgaattaaatattttttaaaaaaatggcttagaacttgtaatctatgcaatatcgcagaatatattttttttagaaaaacatatttactagctagagacatgaaagaaataatatgtgagaataatctggtgagtagctgagaagaacgtggcttgatggtgattggtgagatattaatttgcctatgtttgtggattaggaattccagtatgtaggcccgtacggttggagctacatatgctagccagatcaacttgttttcttctactgtcagagagcttaaactgaattaattaattagctcaaagtcaaagatggtgacactgttaaacacaagtagtttaataaattaattattcaggctggaccgtgtcaagttgttctaataagtatataccttctgggatatctgcagcgatgacggccaactctgccagcagccataggaaaatcttgacgaacttggggtactcactcttgcagatctcagccagatgcctccctaggatgtaaattaatgccggccatatattagcactttatcaagttggtaattacagtttaattgcgtgtcatttagtcacctgtaaccactccaagattagctgctagcgactgtatgataagtgcgaagatgagtccaatcagaatcacccagagcagctgcaaattgaaacaacacaatggacagaaaagcacatagaaatcaattaatggtgcagattattcacttttctcttttctctagatgatgtgagagtgagatacatatgcactggggattaaaagtggttctgaaaagtttgtcaatttggagaatttgatggagctagcagatgaattaatggagtgtgagtctgcgagggacaaggaaagggacgaacacctagaaagcatgaaagttactcatgctttcagtcattcagtgcgtaaacagtaaaataaagtcgactgtacgtacctcatatctgtggttggctccggcttgcagatcggtttccactgtttcattttgtgacaaaattaacagttacaatccagagtttgtcaaaataaaagtttcagttgagagagagagagagagagagagagagagattcagagattctcacaattgccaggatccaagtaggctaaagacaccatgaatccaggaccaacatgggcaaggaaccttttccatgcaggctcctgtacacaatttaccaaaactgagaaactaacaaaaagaaacacagacataattgaggatccaatgttccatcagtccttaattaccacatacacacacacacacatgaacattcatgagcaatgaatgccatgccaggaagtactctcaagatctatctacattaagctctgctcttgcttctgcagttacagtgaacaactgtccatggtgacagtggagtgcttacaagaagacaactttgtgataattaaggtagcaactgaccttcattagcagctgatcatctgcgtcgagcttcttggcatcttgatcatgtgccgcactagctctccagctgatgctccctctctcactgctctctctctcaatctccattgttgcttcctctcttagcttcttcaggctaagctagagctaagctagactcaggctagctggaagtagacgaagaagagaatggtggtagtgacgaactctcaagccacaccctcctgtcctacttatacacaaaccatgcaagtatgcagccaaattaac

SEQ ID NO: 20 - DNA, 5414 bp, Oryza sativa L.
catcagtccactcatccccatgcccccctcattctgatcctttataaatcagccacccagcaagacattcctttccaacctcactcaccttgctctagctcagctagctcttgctgctagtagcttcagctttgctactgctactaccactactactactactactttcacctccaagaaagtatagattgcaagaacaatttctgctgctgttgttctttcttgctgttctttctccttgtgtagttgtgtggtgtggttgagctgaggtgtagttaggacagtggctggctttgcttgcttgattggtcacctgggaaggtcgctgtctctgccttttgttttctttgccatctctttctttctgggctgctttcaccttttgctttccaacctcttcttgccatcaataatgatggaggagtaagatgagggcttgttcttgaagtctctccaatctaatctcatttcatcttcatcatctccacccggtccaaatccacctactactgtttttcttttgtcccttctcttttttttcttcctctgatcttttcttttttcaggtgttttgtttttggcagtaggtgcttgctatcttttcattaggctttggattttcctctcgtgttctttagtctcagcctattagaggtttgaggaaagttttccatttcttagctctttttgcttccagcttcttgggattgctctgtttcttgctgagaattggctcgatttgggaggcctcatcttgtgttagctttcaggttttcttgcctgaaattgaaccgattgggtaggtttttaggtgcttcagtttcagttgctagctcgtcaggcttgttcttgggctcctgccttgaattggggtcaaattgggatgcttctgggtgctttgaggatggaggagaggaagggattgatggggagggagagggatcaattccccgtcggcatgcgggtcctcgccgtcgacgatgacccggtgtgcctcaaggttcttgagaccctcctccggcgctgccaataccatggtcagtatttacagtaaccgcagctaattgtacttccattatcagctatccatgatgctcaacttgctggtttttttgttcgattctgcatgttaatgacagtgttgttcttttgcttctgcttgcattgttcattgttgtattggagtactgtagttttcttagaaatctgaaatgtcactgatactagcatacttggcacattttagaacttctgtatatcttataaatgcaatgcaatatgcctactatggtgcactgataagagctctcttatggtgatttttaacagtaacatcaaccaaccaggctattactgcgttgaagctgctcagggagaacagggacatgtttgatcttgtcatcagtgatgtccacatgcccgacatggacggatttaagctccttgagcttgtggggcttgaaatggatctcccagtcatcagtaagttcagattttcattttttccccttagtacaaacaaagttctagataggaaggatcagctgttgtgtaaaaatgtcttagctgaatttgagcaaaagaaatggagatgctaatcgggtataatgcctgcagtgttatcagtaaatggagagacaaagactgtgatgaagggataactcatggtgcctgtgactatcttctaaaaccggtccgaatcgaagaactaaggaacatatggcagcatgttgttaggaggaagttcggtaatcgtgagcgaaacaatcttgatttctccaaagaatgcaataagccgcaaagcgcggatactgatcatggaccataccaacctacctgtggttcttctgatcaaaatgggaggtccagcaggaaaaggaaagaactacacggcgaggacgacgatgaaggcgatgataatgattatcaagaaaatgatgagccctcagctgcaaagaagcccagagttgtatggtcagttgagctgcaccgaaaatttgttgccgctgtcaaccagcttggaattgacagtaagaaagcacccccccccccaccccacttttttgcctttctgtaaatgtccttgcaggtttgcagcaaactcatttatgtttgtgtcctttttttttttttacctcactgttgatgcagaagctgtaccaaaaagaattcttgagcttatgaatgtggagaaactcaccagggaaaatgttgcaagtcatctacaggtatagcatttacttccatgacaaaattatctgggtgccttttttttttcttttctttttatccattcatgagagctcttgaattttgctcaaccgtcatcctttacatgttcttctgtaccttcagttgttgatgcgtttttacttcagtttatgaggtcgtccatcattcattcattcatcaggcctgcacaaagtgacagaaaatctcttatcattatacatgttgcaaatgctgatgctgcacagcatcgagaattcaagattccaccacatggttgtctgaactctgaaaaactcaccattttatcacatcatttactcaacctgcacagaaatgtagtacaacaaacttcagatcaggattgatccacacagactgcacattttcttccctgatgcaaaattatcatcgatcccgatgaacaccatcgaatcaaagcttcatccaccatttgatttcatcaatgaaatggaggagtctgcacctgagcacacacggccacacagcatatgtagctgggtagaccttcactctaccagagtaagcgatccaggcggcctcgatggcgccgccgcggctcaaactggccggcctcgtcgctgtggcagcgggccagctaggggatggcggcggcgcaggggctccggcgatgggatctgaatccaaacagcagagagggttttggttgagttgccactgaactgaacctgcatatattttggtcatccatatctgaaaaatgtgcaatacttcagatgtttagatcagtatttcctccaaattcttgcaaatattttcttcggctcacgaaattcttgttagcattgttggttgtgaagtggcacacctataatcatgtttgaatttgactcagtctctcatcaccttttatttagtgttgatgcagtaagagtactagaacagagctgatgtttcctactactttcatggctagctagaattttatggtgtcatacaggataaattgaacgaggttccaccattttatctttgttgttcatatgcaactagattttattaaactgttgaaatgcaagaatgtgcaaacatctcgtatgacaaatcctaaacaatgtgctatctaaatctgaaaataggtgatttgctgtttaaaaactcaagtccatatctaatcagttgattttcatgtgacatgcagaagtacaggctttacctcaagagactaggtgctgtagcatcacaacaagccagcattgttgctgcctttggaggcagagatccctccttcttgcatattggagcatttgaaggactccagagctatcaaccttttgcaccttctgctgctcttccatctttcaatccacatggcctgctaacccgaactagcgccgccgcggctttcggacttcaggagcttgctgccccctccagcacaattcagacttctacaggaaatgtcacagttggccattgcttggaagaaaaccagcaggcaaatctagcacaaggcttgaccgcggcgatcgggcaacctcagcttcaacagaactggattcatcaagaaggtaatggtctgtctgatgttttttctgggagttctctgaccaacactttgtccagcacactccaaagagttccaagcagttcattgccaccacaagaactcttggagtgcaaacaagccaaagttagcatgccgccatcgatacggataccgccttctagttcagcacttcttgagaggactcttggggtttccaccaatttgggagattctagtatatcccagcagggtgctcttccaatagatggtggattttctgctgacaggttaccattgcacagttcatttgatggcgctgttgcaacaaagctagatactagtttggcagcttcacagagagagattggccagcaggggaaattttcagttagcatgcttgtctccccttctgacaatcttgcattagccaaaaatgccaaaactggagctagttcttctggcagtactataattctccctcttgatactgcaagacattcagactacttgcagttcggaggtgcaagcaattctttgcagaaaatggatggacagaaacaagatcatatacagagctcaaacattatatggagttcaatgccaagcactcaactgccaagtgatacccaaattcataatactcaaaaccaaagattggacagcggaagttttaaccataatattggtgcccatttggctgaccaaacaaatgcaagtgcgtcaatacttccgcaaatgaagtttgacacaagaatatcagaagagaaaatgaagcagaagaatacatatgacttgggtagttcaaagctgcagggtggatttaattctagtggctgcaattttgatggccttctcaattccataatcaaagtggtatgaacctctaattttcctttcctttttccccatatagttttaaatatttatctacatgaatatgctcttccttctcattgagtaatttaaattgactccactgtatttaagcacaaagaacaaaggccactctaaatattaagctgtaaacagttgtaacctacacataaacaggatatgcctagcagtacctaatatttgcattttccatcttgttttatattatagaaatgttcttgagccacatttaacttggttctaacttatctaatctccatgggactttaccttttgcatatattcttctgaaagaaatgatggtttactgaagccttaattctaattccaatgcaggagaaggatgatctcccattcatggacaatgaattgggctgtgacctttttccacttggtgcctgcatatgaatccatcatttcggacaccaaagattttgcataatctaaaagagtcagctgtgctgcctgatctgtgctacttcatccaggctttgtgtatactaggatctagggacagtagtggcttgtaatcttgctttatctcttctctagggttggctatggaagtatggttagatcaagataagaagaaaatataatgctgtaatgtcagcgccttttctagtcaaaatctgtgtttgtttctaaccaagcaaccacatctagtgtttgctgcaccttctcagactgaaaaaatatggaaactcaccatggcatagataaatcaaaagggaagtctttaaccatccaagtttgctac

SEQ ID NO: 21 - DNA, 5408 bp, Oryza sativa L.
catcagtccactcatccccatgcccccctcattctgatcctttataaatcagccacccagcaagacattcctttccaacctcactcaccttgctctagctcagctagctcttgctgctagtagcttcagctttgctactgctactaccactactactactactactttcacctccaagaaagtatagattgcaagaacaatttctgctgctgttgttctttcttgctgttctttctccttgtgtagttgtgtggtgtggttgagctgaggtgtagttaggacagtggctggctttgcttgcttgattggtcacctgggaaggtcgctgtctctgccttttgttttctttgccatctctttctttctgggctgctttcaccttttgctttccaacctcttcttgccatcaataatgatggaggagtaagatgagggcttgttcttgaagtctctccaatctaatctcatttcatcttcatcatctccacccggtccaaatccacctactactgtttttcttttgtcccttctcttttttttcttcctctgatcttttcttttttcaggtgttttgtttttggcagtaggtgcttgctatcttttcattaggctttggattttcctctcgtgttctttagtctcagcctattagaggtttgaggaaagttttccatttcttagctctttttgcttccagcttcttgggattgctctgtttcttgctgagaattggctcgatttgggaggcctcatcttgtgttagctttcaggttttcttgcctgaaattgaaccgattgggtaggtttttaggtgcttcagtttcagttgctagctcgtcaggcttgttcttgggctcctgccttgaattggggtcaaattgggatgcttctgggtgctttgaggatggaggagaggaagggattgatggggagggagagggatcaattccccgtcggcatgcgggtcctcgccgtcgacgatgacccggtgtgcctcaaggttcttgagaccctcctccggcgctgccaataccatggtcagtatttacagtaaccgcagctaattgtacttccattatcagctatccatgatgctcaacttgctggtttttttgttcgattctgcatgttaatgacagtgttgttcttttgcttctgcttgcattgttcattgttgtattggagtactgtagttttcttagaaatctgaaatgtcactgatactagcatacttggcacattttagaacttctgtatatcttataaatgcaatgcaatatgcctactatggtgcactgataagagctctcttatggtgatttttaacagtaacatcaaccaaccaggctattactgcgttgaagctgctcagggagaacagggacatgtttgatcttgtcatcagtgatgtccacatgcccgacatggacggatttaagctccttgagcttgtggggcttgaaatggatctcccagtcatcagtaagttcagattttcattttttccccttagtacaaacaaagttctagataggaaggatcagctgttgtgtaaaaatgtcttagctgaatttgagcaaaagaaatggagatgctaatcgggtataatgcctgcagtgttatcagtaaatggagagacaaagactgtgatgaaggggataactcatggtgcctgtgactatcttctaaaaccggtccgaatcgaagaactaaggaacatatggcagcatgttgttaggaggaagttcggtaatcgtgagcgaaacaatcttgatttctccaaagaatgcaataagccgcaaagcgcggatactgatcatggaccataccaacctacctgtggttcttctgatcaaaatgggaggtccagcaggaaaaggaaagaactacacggcgaggacgacgatgaaggcgatgataatgattatcaagaaaatgatgagccctcagctgcaaagaagcccagagttgtatggtcagttgagctgcaccgaaaatttgttgccgctgtcaaccagcttggaattgacagtaagaaagcacccccccccccaccccacttttttgcctttctgtaaatgtccttgcaggtttgcagcaaactcatttatgtttgtgtcctttttttttttttacctcactgttgatgcagaagctgtaccaaaaagaattcttgagcttatgaatgtggagaaactcaccagggaaaatgttgcaagtcatctacaggtatagcatttacttccatgacaaaattatctgggtgccttttttttttcttttctttttatccattcatgagagctcttgaattttgctcaaccgtcatcctttacatgttcttctgtaccttcagttgttgatgcgtttttacttcagtttatgaggtcgtccatcattcattcattcatcaggcctgcacaaagtgacagaaaatctcttatcattatacatgttgcaaatgctgatgctgcacagcatcgagaattcaagattccaccacatggttgtctgaactctgaaaaactcaccattttatcacatcatttactcaacctgcacagaaatgtagtacaacaaacttcagatcaggattgatccacacagactgcacattttcttccctgatgcaaaattatcatcgatcccgatgaacaccatcgaatcaaagcttcatccaccatttgatttcatcaatgaaatggaggagtctgcacctgagcacacacggccacacagcatatgtagctgggtagaccttcactctaccagagtaagcgatccaggcggcctcgatggcgccgccgcggctcaaactggccggcctcgtcgctgtggcagcgggccagctaggggatggcggcggcgcaggggctccggcgatgggatctgaatccaaacagcagagagggttttggttgagttgccactgaactgaacctgcatatattttggtcatccatatctgaaaaatgtgcaatacttcagatgtttagatcagtatttcctccaaattcttgcaaatattttcttcggctcacgaaattcttgttagcattgttggttgtgaagtggcacacctataatcatgtttgaatttgactcagtctctcatcaccttttatttagtgttgatgcagtaagagtactagaacagagctgatgtttcctactactttcatggctagctagaattttatggtgtcatacaggataaattgaacgaggttccaccattttatctttgttgttcatatgcaactagattttattaaactgttgaaatgcaagaatgtgcaaacatctcgtatgacaaatcctaaacaatgtgctatctaaatctgaaaataggtgatttgctgtttaaaaactcaagtccatatctaatcagttgattttcatgtgacatgcagaagtacaggctttacctcaagagactaggtgctgtagcatcacaacaagccagcattgttgctgcctttggaggcagagatccctccttcttgcatattggagcatttgaaggactccagagctatcaaccttttgcaccttctgctgctcttccatctttcaatccacatggcctgctaacccgaactagcgccgccgcggacttcaggagcttgctgccccctccagcacaattcagacttctacaggaaatgtcacagttggccattgcttggaagaaaaccagcaggcaaatctagcacaaggcttgaccgcggcgatcgggcaacctcagcttcaacagaactggattcatcaagaaggtaatggtctgtctgatgttttttctgggagttctctgaccaacactttgtccagcacactccaaagagttccaagcagttcattgccaccacaagaactcttggagtgcaaacaagccaaagttagcatgccgccatcgatacggataccgccttctagttcagcacttcttgagaggactcttggggtttccaccaatttgggagattctagtatatcccagcagggtgctcttccaatagatggtggattttctgctgacaggttaccattgcacagttcatttgatggcgctgttgcaacaaagctagatactagtttggcagcttcacagagagagattggccagcaggggaaattttcagttagcatgcttgtctccccttctgacaatcttgcattagccaaaaatgccaaaactggagctagttcttctggcagtactataattctccctcttgatactgcaagacattcagactacttgcagttcggaggtgcaagcaattctttgcagaaaatggatggacagaaacaagatcatatacagagctcaaacattatatggagttcaatgccaagcactcaactgccaagtgatacccaaattcataatactcaaaaccaaagattggacagcggaagttttaaccataatattggtgcccatttggctgaccaaacaaatgcaagtgcgtcaatacttccgcaaatgaagtttgacacaagaatatcagaagagaaaatgaagcagaagaatacatatgacttgggtagttcaaagctgcagggtggatttaattctagtggctgcaattttgatggccttctcaattccataatcaaagtggtatgaacctctaattttcctttcctttttccccatatagttttaaatatttatctacatgaatatgctcttccttctcattgagtaatttaaattgactccactgtatttaagcacaaagaacaaaggccactctaaatattaagctgtaaacagttgtaacctacacataaacaggatatgcctagcagtacctaatatttgcattttccatcttgttttatattatagaaatgttcttgagccacatttaacttggttctaacttatctaatctccatgggactttaccttttgcatatattcttctgaaagaaatgatggtttactgaagccttaattctaattccaatgcaggagaaggatgatctcccattcatggacaatgaattgggctgtgacctttttccacttggtgcctgcatatgaatccatcatttcggacaccaaagattttgcataatctaaaagagtcagctgtgctgcctgatctgtgctacttcatccaggctttgtgtatactaggatctagggacagtagtggcttgtaatcttgctttatctcttctctagggttggctatggaagtatggttagatcaagataagaagaaaatataatgctgtaatgtcagcgccttttctagtcaaaatctgtgtttgtttctaaccaagcaaccacatctagtgtttgctgcaccttctcagactgaaaaaatatggaaactcaccatggcatagataaatcaaaagggaagtctttaaccatccaagtttgctac

SEQ ID NO: 22 - DNA, 46 bp, Artificial Sequence
  Synthetic
gaaggtgaccaagttcatgctagcatgtgttgcctatgatccctag

SEQ ID NO: 23 - DNA, 46 bp, Artificial Sequence
  Synthetic
gaaggtcggagtcaacggattagcatgtgttgcctatgatccctaa

SEQ ID NO: 24 - DNA, 24 bp, Artificial Sequence
  Synthetic
tcaatacacagtcctgccatcacc

SEQ ID NO: 25 - DNA, 42 bp, Artificial Sequence
  Synthetic
gaaggtgaccaagttcatgctcgagtccactgtggaggttcc

SEQ ID NO: 26 - DNA, 42 bp, Artificial Sequence
  Synthetic
gaaggtcggagtcaacggattcgagtccactgtggaggttct

SEQ ID NO: 27 - DNA, 22 bp, Artificial Sequence
  Synthetic
acccctcgatctctatacggtg

SEQ ID NO: 28 - DNA, 18 bp, Oryza sativa L.
cctacgtggcaattcaca

SEQ ID NO: 29 - DNA, 22 bp, Oryza sativa L.
ccaaccaggtgctaagctcatc

SEQ ID NO: 30 - DNA, 34 bp, Oryza sativa L.
tctccccctactgtacaccaccaaaagtggaggg

(19) United States
(12) Patent Application Publication
(10) Pub. No.: US 2022/0348913 A1
(43) Pub. Date: Nov. 3, 2022

(54) METHOD FOR IDENTIFYING M1 GENERATION PLANT MUTANTS RESULTING FROM PHYSICAL AND CHEMICAL MUTAGENESIS AND FOR ACQUIRING MUTANT, IDENTIFICATION OF GENOTYPING PRIMER FOR ORYZA SATIVA MUTATION, MUTANT GENE, AND USE THEREOF

(71) Applicant: HUNAN HYBRID RICE RESEARCH CENTER, Changsha, Hunan (CN)

(72) Inventors: Bingran Zhao, Changsha, Hunan (CN); Ye Shao, Changsha, Hunan (CN); Bigang Mao, Changsha, Hunan (CN); Li Tang, Changsha, Hunan (CN); Yan Peng, Changsha, Hunan (CN); Yuanyi Hu, Changsha, Hunan (CN); Wenjian Li, Changsha, Hunan (CN); Lixia Yu, Changsha, Hunan (CN); Yan Du, Changsha, Hunan (CN); Yaokui Li, Changsha, Hunan (CN); Dan Zhang, Changsha, Hunan (CN); Lianyang Bai, Changsha, Hunan (CN); Longping Yuan, Changsha, Hunan (CN)

(21) Appl. No.: 17/830,731

(22) Filed: Jun. 2, 2022

Related U.S. Application Data
(63) Continuation of application No. PCT/CN2020/077242, filed on Feb. 28, 2020.

(30) Foreign Application Priority Data
Dec. 3, 2019 (CN) 201911222439.8
Dec. 3, 2019 (CN) 201911223356.0
Dec. 3, 2019 (CN) 201911223359.4
Dec. 3, 2019 (CN) 201911223360.7

(51) Int. Cl. C12N 15/11; C12Q 1/6895; C12Q 1/6848; C07K 14/415

(52) U.S. Cl. CPC C12N 15/111; C12Q 1/6895; C12Q 1/6848; C07K 14/415

(57) ABSTRACT

The present disclosure discloses a method for identifying a M1 generation plant mutant resulting from physical and chemical mutagenesis, a method for acquiring the plant mutant, a mutant gene, and use thereof. The method for identifying a M1 generation plant mutant resulting from physical and chemical mutagenesis includes: mutagenizing a plant to obtain an M1 generation plant mutant, extracting a mixed pool of DNA from the obtained M1 generation plant mutant, subjecting the mixed pool of DNA to high-depth targeted sequencing for a target gene region, and aligning a sequencing result with the target gene region to identify whether there are target single nucleotide polymorphisms (SNPs) and/or Indel. The method of the present disclosure has high efficiency and high accuracy, involves simple operations, and is of progressive significance for the identification and acquisition of innovative germplasms.

### CROSS REFERENCE TO RELATED APPLICATION

The present application is a continuation application of PCT application No. PCT/CN2020/077242 filed on Feb. 28, 2020, which claims the benefit of Chinese Patent Application Nos. 201911223356.0, 201911222439.8, 201911223360.7 and 201911223359.4, all filed on Dec. 3, 2019, each of which is incorporated by reference herein in its entirety.

### REFERENCE TO SEQUENCE LISTING

The Sequence Listing is submitted as an ASCII formatted text file via EFS-Web, with a file name of “Sequence_Listing.TXT”, a creation date of Jun. 2, 2022, and a size of 29,316 bytes. The Sequence Listing filed via EFS-Web is part of the specification and is incorporated in its entirety by reference herein.

### TECHNICAL FIELD

The present disclosure belongs to the field of plant mutant identification, and in particular to a method for identifying a plant mutant resulting from physical and chemical mutagenesis, a method for acquiring the plant mutant, a related genotyping primer for mutation, an acquired Oryza sativa mutant gene, and use thereof.

### BACKGROUND

Among cereal crops that are propagated through seeds, Triticum aestivum and Oryza sativa are the main food crops in China. With the rapid development of the national economy and society, the contradiction between population growth and arable land reduction has become increasingly acute. China has a large area of soil with varying salt contents, and the breeding of cereal crops with improved salt tolerance can increase an arable land area and increase a total grain output. Among cereal crops, Oryza sativa has poor salt tolerance, and thus it is urgent to breed new varieties with improved salt tolerance through genetic improvement. Mutagenesis is usually adopted to screen out mutant materials with improved salt tolerance from the Oryza sativa offspring. The cross breeding method can be used to screen out an excellent plant line with recombinant traits and improved salt tolerance, and there are also reports on the cultivation of a plant line with improved salt tolerance through anther culture. There is an urgent need in prior art to acquire germplasm resources with salt tolerance that can be planted in large quantities on coastal saline soils in China, thereby effectively utilizing the limited soil resources. In addition, in recent years, with the frequent occurrence of excessive cadmium in rice, the quality of rice has also become a focus of public attention. The heavy metal Cd is a highly-toxic cumulative nephrotoxicant and carcinogen, and rice Cd is the main source for Cd intake of populations with rice as a staple food. Chinese patent CN105052641A discloses a screening method for low-cadmium Oryza sativa, where Xiangwan Indica No. 12 is selected as a control; a double experiment of field planting and pot planting is conducted; the light, medium, and heavy cadmium pollution are set as three screening conditions; and a low-cadmium Oryza sativa variety/line screened out has comprehensive and stable low-cadmium characteristics. Although there are reports on the selective breeding methods of low-cadmium Oryza sativa in the prior art, these methods mainly focus on the variety screening under environmental stress conditions, and there are few reports on the screening of low-cadmium Oryza sativa by mutation breeding and molecular breeding. Therefore, enhancing the selective breeding of new Oryza sativa varieties with low cadmium accumulation will be conducive to promoting the industrial development of safe and nutritious agricultural products.

Germplasm resources are the basis of plant genetics and breeding. Mutation breeding can produce abundant genetic variations, with a mutation rate thousands of times a mutation rate of natural variation. According to the statistics of the International Atomic Energy Agency (IAEA) in 1985, more than 500 varieties had been bred in countries worldwide through mutagenesis, and a large number of valuable germplasm resources had been obtained. However, mutation breeding also has many obvious defects. Most importantly, a mutation position is random and a target mutant can only be selected in an M2 generation, which leads to the low accuracy and efficiency for innovative germplasms.

With the popularization of gene editing technology in plants, this technology effectively makes up for the defects of mutation breeding with its characteristics of accuracy, efficiency, and convenience, and is favored by the plant genetics and breeding community. Although the gene editing technology overcomes the main shortcomings of mutation breeding, the application of gene editing technology in plants requires transgenesis, and some important plant transgenic systems are immature, which limits the application of gene editing technology. Mutation breeding is achieved through physical and chemical means without the aid of transgenesis, and a stable genetic mutant germplasm can be obtained from most plants through mutagenesis. Therefore, mutation breeding still has irreplaceable advantages. In addition, the application of mutation breeding in plants has a long history. In 1927, at the Third International Congress of Human Genetics, Muller discussed that X-rays could induce Drosophila melanogaster (D. melanogaster) to produce a large number of variations, and proposed the induced mutation to improve plants. Subsequently, Stadler demonstrated for the first time that X-rays could induce mutations in Zea mays and Hordeum vulgare. Nilsson-Ehle and Gustafsson (1930) used X-ray irradiation to obtain a standup Hordeum vulgare mutant with hard stalks and compact panicles. In 1934, Tollenear bred the first Nicotiana tabacum mutant variety “Chlorina” using X-rays. In 1948, a drought-resistant Gossypium variety was bred through X-ray mutagenesis in India. In 1957, the first Institute for Agricultural utilization of Atomic Energy in China was established by the Chinese Academy of Agricultural Sciences, and then relevant research institutes were successively established in various provinces. In the mid-1960s, new varieties were bred through irradiation mutagenesis for major crops such as Oryza sativa, Triticum aestivum, and Glycine max, and were used in production. In the late 1970s, the plant irradiation mutation breeding began to be used in the breeding of vegetables, sugar crops, melons and fruits, feeds, and medicinal and ornamental plants. It can be seen that the mutation breeding has been tested for a long time and has made great contributions to the genetic breeding of plants worldwide; and the mutation breeding has become a generally-accepted breeding method in both scientific and industrial circles.

For a long time, the general knowledge in mutation breeding is that mutation selection is not conducted in the mutagenized generation (M1), because the genetic variation caused by mutagenesis is mostly recessive and exists in the form of chimera in M1. Only in the segregating population of the second generation of mutagenesis (M2), the recessive variation produced in M1 will exist in the form of homozygosity in M2 individuals, among which a mutant with a target trait can be selected. In view of the irreplaceable advantages of mutation breeding, it is expected to solve the problem that the efficiency of selecting beneficial mutations and target gene mutations is low.

### SUMMARY

The technical problem to be solved by the present disclosure: In order to overcome the deficiencies and defects mentioned in the above background, the present disclosure provides a high-efficiency, high-accuracy, and convenient method for high-throughput targeted identification of an M1 generation plant mutant resulting from physical and chemical mutagenesis, a method for acquiring the mutant with less save manpower and material resources, high efficiency, and high accuracy, a variety of Oryza sativa mutant genes, and a KASP genotyping primer for detecting, screening, or acquiring a mutant carrying an Oryza sativa mutant gene.

In order to solve the above-mentioned technical problem, the present disclosure provides a method for high-throughput targeted identification of an M1 generation plant mutant resulting from physical and chemical mutagenesis, including the following steps:

a) mutagenizing plants by the physical and chemical mutagenesis at a non-lethal dose to obtain an M1 generation plant material;

b) planting each individual plant in the M1 generation plant material independently, collecting leaves from each planted individual plant, and mixing leaves collected from an individual plant;

c) extracting a mixed pool of DNA from a mixed leaf material;

d) subjecting the mixed pool of DNA to high-depth targeted sequencing for a target gene region; and

e) aligning a high-depth targeted sequencing result with a related sequence of the target gene region to identify whether a population DNA sample in the high-depth targeted sequencing result includes target single nucleotide polymorphisms (SNPs) and/or Indels of the target gene region.

The next-generation sequencing (NGS) and the latest third-generation sequencing have accelerated the research in the fields of genetic diseases, cancer, and the like, and have been gradually used in clinics as an advanced genetic test method. Among various sequencing technologies, the targeted sequencing technology is finally selected after the full consideration of flexibility and low cost. Innovative plant germplasms are rapidly screened and identified through high-depth sequencing of a target region of a genome, which greatly improves the accuracy and efficiency of existing innovative germplasm screening.

In the above method, preferably, after the identification in step e) is completed, the method may further include using one or more verification modes to verify an identification result, such as to determine whether the sample includes the target SNPs and/or Indels of the target gene region; and the verification mode may specifically include:

e₁: testing all plants by a digital polymerase chain reaction (dPCR) identification mode;

e₂: genotyping each individual plant by a Kompetitive Allele Specific PCR (KASP) genotyping mode for verification; and

e₃: verifying each individual plant by a Sanger sequencing mode.

dPCR is a new generation of PCR technology rapidly developed in recent years, and is an absolute quantification technology for nucleic acids. With ultra-high sensitivity, a dPCR system can easily quantitatively analyze low-frequency mutations as low as 0.01%. dPCR is an absolute quantification technology accurate to a single DNA molecule and thus has ultra-high accuracy. With the combination of dPCR and high-depth targeted sequencing, the low-frequency mutations detected by deep sequencing can be accurately verified, which further improves the accuracy and efficiency of existing innovative germplasm screening.

KASP is a high-throughput known SNP/Indel detection technology based on terminal fluorescence reading, where different genotypes at a same locus can be detected through two-color fluorescence. The high-throughput genotyping and identification of different individuals is conducted with the KASP technology for SNPs/Indels discovered by high-depth targeted sequencing to further improve the accuracy and efficiency of existing innovative germplasm screening.

In the above method, more preferably, in step e), the specific identification mode may include the following two steps:

1) detecting population DNA in the high-depth targeted sequencing result by the dPCR identification mode to identify whether the population DNA sample includes target SNPs and/or Indels of the target gene region, and if so, proceeding to step 2), otherwise, finishing; and

2) based on SNP and/or Indel loci identified by dPCR, designing a KASP genotyping primer, and subjecting each individual plant in a population corresponding to a mixed pool sample of the plant mutant to KASP genotyping to finally determine whether there is a chimeric individual plant with a mutation in the target gene region.

KASP is a high-throughput known SNP/Indel detection technology based on terminal fluorescence reading, where different genotypes at a same locus can be detected through two-color fluorescence. dPCR is used to narrow down a population range in a candidate zone and then different individuals are accurately genotyped with the KASP technology for SNPs/Indels discovered by high-depth targeted sequencing to further improve the accuracy and efficiency of existing innovative germplasm screening.

In the above method, preferably, the related sequence of the target gene region may be a gene sequence of Oryza sativa OsNramp5 gene, there may be a 18 bp deletion of [CTACGTGGCAATTCACA (SEQ ID NO: 28)/−] at 8875646-8875663 from a junction between intron 8 and exon 9 of the OsNramp5 gene, and the KASP genotyping primer may include the following sequences:

FAM
(SEQ ID NO: 1)
5’-GAAGGTGACCAAGTTCATGCTGAAGAACCTGCACCCGTCCT-3’,
HEX
(SEQ ID NO: 2)
5’-GAAGGTCGGAGTCAACGGATTGAAGAACCTGCACCCGTCAC-3’,
and
COMMON
(SEQ ID NO: 3)
5’-GCATGGAAAGAAACTGAACAAAGAT-3’.

In the above method, preferably, the related sequence of the target gene region may be a gene sequence of Oryza sativa OsRR22 gene, there may be a 1 bp deletion of [G/−] at 4138902 in exon 3 of the OsRR22 gene, and the KASP genotyping primer may include the following sequences:

FAM
(SEQ ID NO: 4)
5’-GAAGGTGACCAAGTTCATGCTCAGGCACCATGAGTTATCCCT-3’,
HEX
(SEQ ID NO: 5)
5’-GAAGGTCGGAGTCAACGGATTCAGGCACCATGAGTTATCCCC-3’,
and
COMMON
(SEQ ID NO: 6)
5’-TGTTATCAGTAAATGGAGAGACAAAGAC-3’.

In the above method, preferably, the related sequence of the target gene region may be a gene sequence of Oryza sativa OsRR22 gene, there may be a 7 bp deletion of [CGGCTTT/−] at 4140861-4140867 in exon 5 of the OsRR22 gene, and the KASP genotyping primer may include the following sequences:

FAM
(SEQ ID NO: 7)
5’-GAAGGTGACCAAGTTCATGCTGCAAGCTCCTGAAGTCCGAA-3’,
HEX
(SEQ ID NO: 8)
5’-GAAGGTCGGAGTCAACGGATTCAAGCTCCTGAAGTCCGCG-3’,
and
COMMON
(SEQ ID NO: 9)
5’-TTCTGCTGCTCTTCCATCTTTCA-3’.

In the above method, preferably, the non-lethal dose in step a) may refer to controlling a dose within a range of 20% higher and lower a median-lethal dose. The dose controlled within this range can not only achieve a specified mutation rate, but also lead to a specified number of live seeds. For example, a median-lethal dose is particularly preferred. A balance relationship between the mutation efficiency and the number of live seeds can be coordinated by controlling the dose of mutagenesis.

In the above method, preferably, the physical and chemical mutagenesis in step a) may include one or two selected from the group consisting of physical mutagenesis and chemical mutagenesis;

the physical mutagenesis may include ultraviolet (UV) mutagenesis, X-ray mutagenesis, γ-ray mutagenesis, β-ray mutagenesis, α-ray mutagenesis, high-energy particle mutagenesis, cosmic ray mutagenesis, and microgravity mutagenesis, and the physical mutagenesis is more likely to induce Indel mutation;

the chemical mutagenesis may include alkylating agent mutagenesis, azide mutagenesis, base analog mutagenesis, lithium chloride mutagenesis, antibiotic mutagenesis, and intercalative dye mutagenesis, and the chemical mutagenesis is more likely to induce SNP mutation; and

the alkylating agent mutagenesis may include ethyl methanesulfonate (EMS) mutagenesis, diethyl sulfate (DES) mutagenesis, and ethyleneimine (EI) mutagenesis.

In the above method, preferably, in step b), when each individual plant in the M1 generation plant material is planted independently, an arbitrary number of plants may be clustered as a population, and each population may be numbered; and in step c), leaves of each population may be mixed in a centrifuge tube for DNA extraction, such that each tube of DNA includes the genetic information of the entire population. A large sample size is enabled in the sequencing through this grouping operation, a large number of samples can be pooled to reduce the cost of sequencing, and the KASP technology is subsequently adopted for genotyping, such as to finally achieve an effective balance between high throughput and low cost.

As a further preference, the population may include 48, 96, or 192 plants; and during the leaf collection, leaves may be collected from different parts of a same individual plant at a same amount. As a further preference, in step d), a sequencing depth of the high-depth targeted sequencing for a single population with 48 plants may be greater than 2,000×, a sequencing depth of the high-depth targeted sequencing for a single population with 96 plants may be greater than 5,000×, and a sequencing depth of the high-depth targeted sequencing for a single population with 192 plants may be greater than 10,000×.

In the above method, preferably, in step d), the target gene region may include an exon region of a target gene or a non-coding region of the target gene (or other regions of interest on a plant genome, where an exon region is particularly preferred); the high-depth targeted sequencing may include multiplex PCR-based targeted capture technology, liquid-phase probe hybridization capture-based targeted capture technology, or third-generation sequencing-based single-molecule targeted sequencing technology; and

a sequencing depth of the high-depth targeted sequencing may be determined according to a number of individual plants in each population.

From the experimental data in the examples of the present disclosure, the method of the present disclosure can be used in various plants such as Oryza sativa, Sorghum bicolor, Zea mays, and even vegetables, and from some experiments conducted in the present disclosure, preferably, the target gene may be the Oryza sativa OsNramp5 gene or Oryza sativa OsRR22 gene. Based on the results of late experiments, it is not excluded that the method can also be used for other genotypes of other plants.

As a general technical idea, the present disclosure also provides an Oryza sativa mutant gene obtained in the identification of an M1 generation Oryza sativa mutant resulting from physical and chemical mutagenesis by the method described above, where the Oryza sativa mutant gene is an Oryza sativa OsNramp5⁻¹⁸ mutant gene, an Oryza sativa OsRR22⁻¹ mutant gene, or an Oryza sativa OsRR22⁻⁷ mutant gene;

compared with the OsNramp5 gene sequence of Nipponbare, the Oryza sativa OsNramp5⁻¹⁸ mutant gene may include a 18 bp deletion of [CCTACGTGGCAATTCACA (SEQ ID NO: 28)/−] at 8875646-8875663 (RAP_Locus) in exon 9;

compared with theOsRR22 gene sequence of Nipponbare, the Oryza sativa OsRR22⁻¹ mutant gene may include a 1 bp deletion of [G/−] at 4138902 (RAP_Locus) in exon 3 of the OsRR22 gene; and

compared with the OsRR22 gene sequence of Nipponbare, the Oryza sativa OsRR22⁻⁷ mutant gene may include a 7 bp deletion of [CGGCTTT/−] at 4140861-4140867 (RAP_Locus) in exon 5.

Among the Oryza sativa mutant genes described above, more preferably, the Oryza sativa OsNramp5⁻¹⁸ mutant gene may have a nucleotide sequence shown in SEQ ID NO: 19, or a truncated sequence of the nucleotide sequence, or a specific sequence that has 95% or more homology with the nucleotide sequence and encodes the same functional protein as the nucleotide sequence;

the Oryza sativa OsRR22⁻¹ mutant gene may have a nucleotide sequence shown in SEQ ID NO: 20, or a truncated sequence of the nucleotide sequence, or a specific sequence that has 95% or more homology with the nucleotide sequence and encodes the same functional protein as the nucleotide sequence; and

the Oryza sativa OsRR22⁻⁷ mutant gene may have a nucleotide sequence shown in SEQ ID NO: 21, or a truncated sequence of the nucleotide sequence, or a specific sequence that has 95% or more homology with the nucleotide sequence and encodes the same functional protein as the nucleotide sequence.

As a general technical idea, the present disclosure also provides use of the Oryza sativa mutant gene described above in molecular marker-assisted breeding (MAB) of a crop.

In the above use, preferably, when the Oryza sativa mutant gene is the Oryza sativa OsNramp5⁻¹⁸ mutant gene, the Oryza sativa OsNramp5⁻¹⁸ mutant gene or a mutant carrying the Oryza sativa OsNramp5⁻¹⁸ mutant gene may be used in the selective breeding or preparation of an Oryza sativa variety with a low-cadmium-absorption phenotype; and

when the Oryza sativa mutant gene is the Oryza sativa OsRR22⁻¹ mutant gene or the Oryza sativa OsRR22⁻⁷ mutant gene, the Oryza sativa OsRR22⁻¹ or OsRR22⁻⁷ mutant gene or a mutant carrying the Oryza sativa OsRR22⁻¹ or OsRR22⁻⁷ mutant gene may be used in the selective breeding or preparation of an Oryza sativa variety with a salt-tolerant phenotype.

As a general technical idea, the present disclosure also provides a method for high-throughput targeted identification of an M1 generation mutant resulting from physical and chemical mutagenesis and for acquisition of the mutant, including: on the basis of identifying a chimeric individual plant with a mutation of a target gene region by the above method of the present disclosure, further conducting the following steps:

f) for each chimeric individual plant with the mutation of the target gene region, extracting DNA of leaves corresponding to each ear for DNA identification, selecting an ear with the mutation, and mixed-collecting seeds; and

g) mixed-sowing seeds (M2) obtained from the mixed-collecting, and collecting leaves from each individual plant independently for DNA identification to finally obtain an M2 individual plant with a target genetic phenotype.

In the above method, preferably, the chimeric individual plant with the mutation of the target gene region may refer to a chimeric individual plant with the Oryza sativa OsNramp5⁻¹⁸ mutant gene, the Oryza sativa OsRR22⁻¹ mutant gene, or the Oryza sativa OsRR22⁻⁷ mutant gene;

compared with the OsNramp5 gene sequence of Nipponbare, the Oryza sativa OsNramp5⁻¹⁸ mutant gene may include a 18 bp deletion of [CCTACGTGGCAATTCACA (SEQ ID NO: 28)/−] at 8875646-8875663 (RAP_Locus) in exon 9;

compared with the OsRR22 gene sequence of Nipponbare, the Oryza sativa OsRR22⁻¹ mutant gene may include a 1 bp deletion of [G/−] at 4138902 (RAP_Locus) in exon 3 of the OsRR22 gene; and

compared with the OsRR22 gene sequence of Nipponbare, the Oryza sativa OsRR22⁻⁷ mutant gene may include a 7 bp deletion of [CGGCTTT/−] at 4140861-4140867 (RAP_Locus) in exon 5.

In the above method, preferably, in step f), the DNA identification may preferably be conducted using the designed KASP genotyping primer, but can also be conducted by Sanger sequencing for the target region; and the use of the above KASP genotyping primer for identification can well achieve high throughput, low cost, and operation convenience.

In step g), the DNA identification may preferably be conducted using the designed KASP genotyping primer, but can also be conducted by Sanger sequencing for the target region; and the use of the above KASP genotyping primer for identification can well achieve high throughput, low cost, and operation convenience.

As a general technical idea, the present disclosure also provides a KASP genotyping primer used for identifying or acquiring an Oryza sativa mutant in the method described above, including any one selected from the group consisting of the following primer pairs:

(1) a primer pair 1 for identifying or acquiring the Oryza sativa OsNramp5⁻¹⁸ mutant gene:

FAM
(SEQ ID NO: 1)
5’-GAAGGTGACCAAGTTCATGCTGAAGAACCTGCACCCGTCCT-3’,
HEX
(SEQ ID NO: 2)
5’-GAAGGTCGGAGTCAACGGATTGAAGAACCTGCACCCGTCAC-3’,
and
COMMON
(SEQ ID NO: 3)
5’-GCATGGAAAGAAACTGAACAAAGAT-3’;

(2) a primer pair 2 for identifying or acquiring the Oryza sativa OsRR22⁻¹ mutant gene:

FAM
(SEQ ID NO: 4)
5’-GAAGGTGACCAAGTTCATGCTCAGGCACCATGAGTTATCCCT-3’,
HEX
(SEQ ID NO: 5)
5’-GAAGGTCGGAGTCAACGGATTCAGGCACCATGAGTTATCCCC-3’,
and
COMMON
(SEQ ID NO: 6)
5’-TGTTATCAGTAAATGGAGAGACAAAGAC-3’;

and

(3) a primer pair 3 for identifying or acquiring the Oryza sativa OsRR22⁻⁷ mutant gene:

FAM
(SEQ ID NO: 7)
5’-GAAGGTGACCAAGTTCATGCTGCAAGCTCCTGAAGTCCGAA-3’,
HEX
(SEQ ID NO: 8)
5’-GAAGGTCGGAGTCAACGGATTCAAGCTCCTGAAGTCCGCG-3’,
and
COMMON
(SEQ ID NO: 9)
5’-TTCTGCTGCTCTTCCATCTTTCA-3’.

Compared with the prior art, the present disclosure has the following advantages:

In the present disclosure, the high-depth targeted sequencing technology is creatively combined with one or more selected from the group consisting of the targeted sequencing technology, the dPCR technology, and the KASP genotyping technology, and a resulting combination is creatively used in the mutation screening for mutation breeding, which can realize the high-throughput and accurate selection of a target gene mutation in a mutagenized M1 generation and accurately locate a chimeric individual plant with the target gene mutation. The technical solution of the present disclosure not only saves the time cost required for the propagation of one generation, but also solves the problem that the large increase in the number of populations after the propagation of one generation makes land, labor, and mutation selection costs too high, which is of important progressive significance for the identification and acquisition of innovative germplasms.

Based on the identification method and screening method of the present disclosure, the present disclosure also creatively acquires the Oryza sativa OsNramp5⁻¹⁸ mutant gene, the Oryza sativa OsRR22⁻¹ mutant gene, and the Oryza sativa OsRR22⁻⁷ mutant gene, and provides use of the above-mentioned mutant genes in the MAB of crops and in the selective breeding or preparation of an Oryza sativa variety with a low-cadmium-absorption phenotype and an Oryza sativa variety with a salt-tolerant phenotype, such that remarkable technical effects are achieved and an Oryza sativa variety with a low-cadmium-absorption phenotype and an Oryza sativa variety with a salt-tolerant phenotype are finally obtained.

In addition, the KASP genotyping primer designed for detecting, screening, or acquiring a mutant carrying the Oryza sativa mutant gene in the present disclosure can be used to accurately genotype different individuals for SNPs/Indels discovered by high-depth targeted sequencing, which can further improve the accuracy and efficiency of screening and identifying innovative germplasms.

The technical solution of the present disclosure not only saves the time cost required for the propagation of one generation, but also solves the problem that the large increase in the number of populations after the propagation of one generation makes land, labor, and mutation selection costs too high, which is of important progressive significance.

### BRIEF DESCRIPTION OF THE DRAWINGS

To describe the technical solutions in examples of the present disclosure or in the prior art more clearly, the accompanying drawings required for describing the examples or the prior art will be briefly described below. Apparently, the accompanying drawings in the following description show some examples of the present disclosure, and a person of ordinary skill in the art may still derive other drawings from these accompanying drawings without creative efforts.

FIG. 1 is a schematic diagram illustrating a process flow of the method for high-throughput targeted identification of an M1 generation plant mutant resulting from physical and chemical mutagenesis and for acquisition of the mutant according to the present disclosure.

FIG. 2 shows a genotyping result of a population 32 by the KASP genotyping primer in Example 1 of the present disclosure.

FIG. 3 shows a genotyping result of a population 26 by the KASP genotyping primer in Example 1 of the present disclosure.

FIG. 4 shows a genotyping result of a population 34 by the KASP genotyping primer in Example 1 of the present disclosure.

FIG. 5 is a comparison photo of seedlings of a mutant Huazhan-Nramp5⁻¹⁸ and Huazhan (wild type (WT)) that are cultivated at the same time in Example 4 of the present disclosure.

FIG. 6 shows a comparison result of a measured dry weight of seeds per plant between a mutant and a control in Example 4 of the present disclosure.

FIG. 7 shows a comparison result of a measured Cd content in brown rice between a mutant and a control in Example 4 of the present disclosure.

FIG. 8 is a comparison photo of seedlings of a mutant Huazhan-OsRR22⁻¹ and Huazhan (WT) that are cultivated at the same time in Example 5 of the present disclosure.

FIG. 9 is a comparison photo of seedlings of a mutant Huazhan-OsRR22⁻⁷ and Huazhan (WT) that are cultivated at the same time in Example 6 of the present disclosure.

### DETAILED DESCRIPTION OF THE EMBODIMENTS

In order to facilitate the understanding of the present disclosure, the present disclosure is described in detail below in conjunction with the accompanying drawings of the specification and the preferred examples, but the protection scope of the present disclosure is not limited to the following specific examples.

Unless otherwise defined, all technical terms used hereinafter have the same meaning as commonly understood by those skilled in the art. The technical terms used herein are merely for the purpose of describing specific examples, and are not intended to limit the protection scope of the present disclosure.

Unless otherwise specified, various raw materials, reagents, instruments, equipment, and the like used in the present disclosure can be purchased from the market or can be prepared by existing methods.

### Example 1

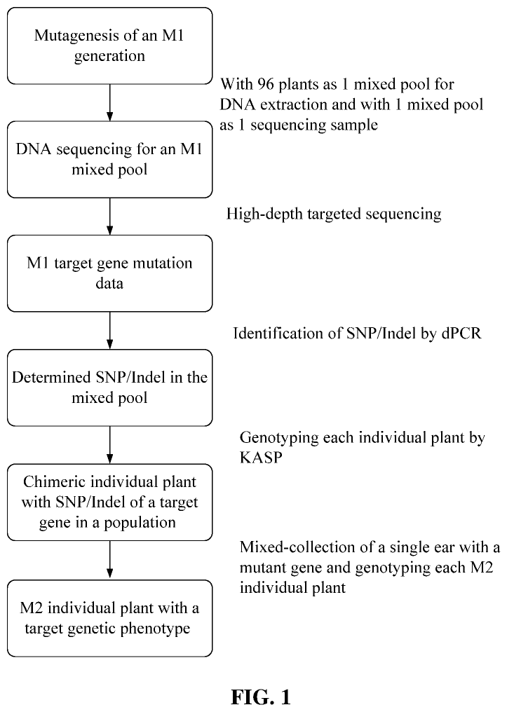

A method for high-throughput targeted identification of an M1 generation plant mutant resulting from physical and chemical mutagenesis and for acquisition of the mutant as shown in FIG. 1 was provided, specifically including the following steps:

1. 20,000 seeds of the Oryza sativa variety 638S (hereinafter referred to as 638S) were irradiated with 80 MeV/u carbon ions (¹²C⁶⁺) at a dose of 180 Gy to obtain an M1 generation of the Oryza sativa variety 638S.

2. Seedlings were cultivated using the 20,000 638S M1 generation seeds, and then each individual seedling was planted independently. 96 plants, as a population, were planted in 8 rows, with 12 plants in each row. 100 populations, a total of 9,600 plants, were planted and numbered 1 to 100.

3. With each population as a unit, leaves were collected from each individual plant at an equal amount, and a total of 96 leaf samples were mixed in a centrifuge tube for DNA extraction. A total of 100 DNA samples were extracted and then numbered corresponding to the population numbers of 1 to 100.

4. With DNA of each population as a sequencing sample, targeted high-depth sequencing was conducted for exon regions of the OsNramp5 (0507g0257200) and OsRR22 (0506g0183100) genes at a sequencing depth of >5,000×.

5. The high-depth targeted sequencing data were aligned with a Nipponbare sequence to obtain the SNP and Indel data of the exons of the OsNramp5 and OsRR22 genes in the 100 samples. According to test results, it was found that sample 32 had an 18 bp deletion of [CCTACGTGGCAATTCACA (SEQ ID NO: 28)/−] at 8875646-8875663 (RAP_Locus) of the OsNramp5 gene, in which 1 bp was at a junction between intron 8 and exon 9; according to test results, it was also found that sample 26 had a 1 bp deletion of [G/−] at 4138902 (RAP_Locus) of the OsRR22 gene, which was located in exon 3; and according to test results, it was also found that sample 34 had a 7 bp deletion of [CGGCTTT/−] at 4140861-4140867 (RAP_Locus) of the OsRR22 gene, which was located in exon 5.

6. The Indels in the sequencing data of the above population samples 32, 26, and 34 were verified by the dPCR technology. Verification results showed that the deletion mutation genotypes in the targeted sequencing data were present in the three samples, respectively.

7. According to the deletion mutation genotypes in the DNA of the population samples 32, 26, and 34, the following KASP genotyping primers were designed (which were produced by Beijing Tsingke Biotechnology Co., Ltd.):

32FAM 5′-GAAGGTGACCAAGTTCATGCTGAAGAACCTGCACCCGTCCT-3′ (as shown in SEQ ID NO: 1, in which an underlined part represents a fluorescent tag primer);

32HEX 5′-GAAGGTCGGAGTCAACGGATTGAAGAACCTGCACCCGTCAC-3′ (as shown in SEQ ID NO: 2, in which an underlined part represents a fluorescent tag primer);

32COMMON 5′-GCATGGAAAGAAACTGAACAAAGAT-3′ (as shown in SEQ ID NO: 3);

26FAM 5′-GAAGGTGACCAAGTTCATGCTCAGGCACCATGAGTTATCCCT-3′ (as shown in SEQ ID NO: 4, in which an underlined part represents a fluorescent tag primer);

26HEX 5′-GAAGGTCGGAGTCAACGGATTCAGGCACCATGAGTTATCCCC-3′ (as shown in SEQ ID NO: 5, in which an underlined part represents a fluorescent tag primer);

26COMMON 5′-TGTTATCAGTAAATGGAGAGACAAAGAC-3′ (as shown in SEQ ID NO: 6);

34FAM 5′-GAAGGTGACCAAGTTCATGCTGCAAGCTCCTGAAGTCCGAA-3′ (as shown in SEQ ID NO: 7, in which an underlined part represents a fluorescent tag primer);

34HEX 5′-GAAGGTCGGAGTCAACGGATTCAAGCTCCTGAAGTCCGCG-3′ (as shown in SEQ ID NO: 8, in which an underlined part represents a fluorescent tag primer); and

34COMMON 5′-TTCTGCTGCTCTTCCATCTTTCA-3′ (as shown in SEQ ID NO: 9).

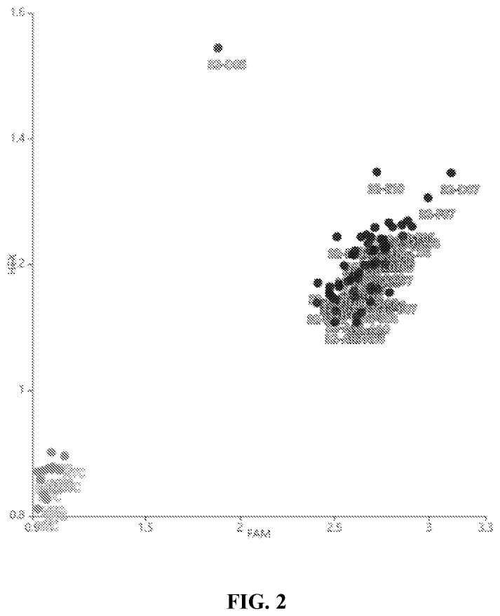

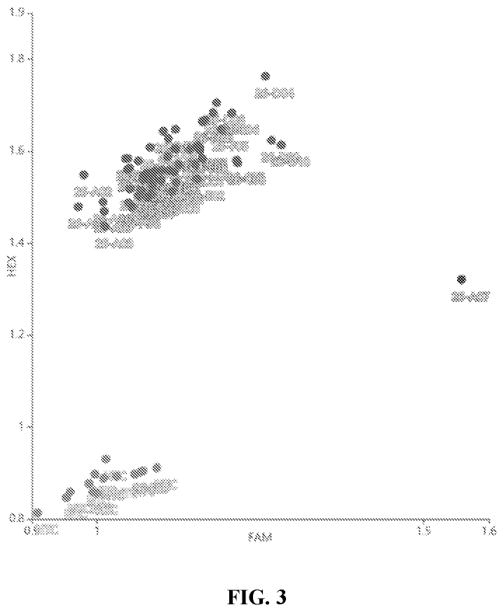

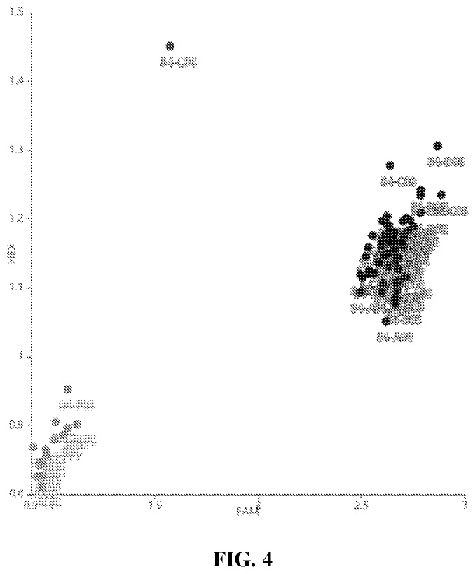

8. The KASP genotyping primers for the 3 deletion mutation loci were used to genotype the 96×3=288 individual plants in the populations 32, 26, and 34. According to genotyping results, it was found that the 5th plant in row 4 of the population 32 (32-D-5) had an 18 bp deletion genotype of [CCTACGTGGCAATTCACA (SEQ ID NO: 28)/−] in exon 9 of the OsNramp5 gene (as shown in FIG. 2); it was also found that the 7th plant in row 1 of the population 26 (26-A-7) had a 1 bp deletion genotype of [G/−] in exon 3 of the OsRR22 gene (as shown in FIG. 3); and it was also found that the 6th plant in row 3 of the population 34 (34-C-6) had a 7 bp deletion genotype of [CGGCTTT/−] in exon 5 of the OsRR22 gene (as shown in FIG. 4). Finally, 3 M1 generation individual plants with a deletion mutation of the target gene were obtained, including 1 chimera 32-D-5 with an OsNramp5 gene frameshift mutation and 2 chimeras 26-A-7 and 34-C-6 with an OsRR22 gene frameshift mutation.

The use of the KASP genotyping primers for the deletion mutation loci to genotype the 96×3=288 individual plants in the populations 32, 26, and 34 was specifically as follows:

(1) extraction of Oryza sativa leaf DNA by the cetyltrimethylammonium bromide (CTAB) method;

(2) 5 μl of a KASP genotyping system:

| | | |
|---|---|---|
| FAM-X 10 μM | 0.15 | μl |
| HEX-Y 10 μM | 0.15 | μl |
| COMMON 10 μM | 0.3 | μl |
| KASP MIX | 2.5 | μl |

| | |
|---|---|
| sample DNA | 10 ng to 100 ng |
| H₂O | making up to 5 μl; |

(3) PCR reaction procedure

| | |
|---|---|
| 94° C. | 10 min |
| 94° C. | 20 s ] 10× |
| 65° C. to 0.8° C./cycle | 1 min |
| 94° C. | 20 s ] 30× |
| 57° C. | 1 min |
| 4° C. | ∞. |

9. DNA was extracted from leaves corresponding to each ear on each of the individual plants 32-D-5, 26-A-7, and 34-C-6, and a sequencing test was conducted for the Indel mutation in each individual plant. It was found that 3 ears on the individual plant 32-D-5 had the mutation genotype (as shown in SEQ ID NO: 19) of [CTACGTGGCAATTCACA (SEQ ID NO: 28)/−], 2 ears on the individual plant 26−A-7 had the mutation genotype of [G/−] (as shown in SEQ ID NO: 20), and 4 ears on the individual plant 34-C-6 had the mutation genotype (as shown in SEQ ID NO: 21) of [GGCTTT/−]. With each individual plant as a unit, seeds of ears with the mutation genotype were mixed-collected.

10. Seeds of each of 32-D-5, 26-A-7, and 34-C-6 were grouped into 3 populations and then sown, and each individual plant was genotyped with the KASP primers designed in step 7 above. Finally, 6385-32-D-5 with a low-cadmium-absorption phenotype was obtained from an M2 population of 32-D-5; and 638S-26-A-7 and 638S-34-C-6 with a salt-tolerant phenotype was obtained from M2 populations of 26-A-7 and 34-C-6, respectively.

### Example 2

A method for high-throughput targeted identification of an M1 generation plant mutant resulting from physical and chemical mutagenesis and for acquisition of the mutant as shown in FIG. 1 was provided, specifically including the following steps:

1. 20,000 seeds of the Oryza sativa variety Huahang 31 were taken and soaked in water for 16 h in a constant-temperature incubator at 28° C., and then taken out and air-dried; the seeds were then soaked in a 1% (w/w) EMS solution for 8 h in a constant-temperature incubator at 28° C., and then taken out and air-dried; and the seeds were rinsed with water 8 times, subjected to germination at 37° C., and sown for seedling cultivation, and resulting seedlings were planted. 96 plants, as a population, were planted in 8 rows, with 12 plants in each row. 100 populations, a total of 9,600 plants, were planted and numbered 1 to 100.

2. With each population as a unit, leaves were collected from each individual plant at an equal amount, and a total of 96 leaf samples were mixed in a centrifuge tube for DNA extraction. A total of 100 DNA samples were extracted and then numbered corresponding to the population numbers of 1 to 100.

3. With DNA of each population as a sequencing sample, targeted high-depth sequencing was conducted for an exon region of the OsBADH2 (0508g0424500) gene at a sequencing depth of >5,000×.

4. The high-depth targeted sequencing data were aligned with a Nipponbare sequence to obtain the SNP and Indel data of the exon of the OsBADH2 gene in the 100 samples. According to test results, it was found that sample 72 had a single base mutation of [G/A] at 20381445 (RAP_Locus) of the OsBADH2 gene, which caused an amino acid 109 to change from Trp to a terminator, such that the protein coding of the OsBADH2 gene was terminated prematurely.

5. The SNP in the sequencing data of the above population sample 72 was verified by the dPCR technology. Verification results showed that the SNP genotype in the targeted sequencing data was present in this sample.

6. According to the SNP genotype in the DNA of the population sample 72, the following KASP genotyping primers were designed:

72FAM 5′-GAAGGTGACCAAGTTCATGCTGAAGCCTCTTGATGAAGCAGCATG-3′ (as shown in SEQ ID NO: 10, in which an underlined part represents a fluorescent tag primer);

72HEX 5′-GAAGGTCGGAGTCAACGGATTGAAGCCTCTTGATGAAGCAGCATA-3′ (as shown in SEQ ID NO: 11, in which an underlined part represents a fluorescent tag primer); and

72COMMON 5′-CAACATCGTCCTGACAAATGGAAT-3′ (as shown in SEQ ID NO: 12).

7. The KASP genotyping primers designed above were used to genotype the 96 individual plants in the population 72. According to genotyping results, it was found that the 3rd plant in row 7 of the population 72 (72-G-3) had the single base mutation genotype of [G/A] in exon 3 of the OsBADH2 gene. Finally, an M1 generation individual plant 72-G-3 with an early termination mutation of the target gene was obtained.

8. DNA was extracted from leaves corresponding to each ear on the individual plant 72-G-3, and a sequencing test was conducted for the SNP mutation in the individual plant. It was found that 3 ears on the individual plant 72-G-3 had the single base mutation genotype of [G/A]. Seeds of ears with the mutation genotype were mixed-collected.

9. Seedlings were cultivated using the seeds of the individual plant 72-G-3, then each individual seedling was planted independently, and each individual plant was genotyped with the KASP primers designed in step 6 above. Finally, Huahang 31-72-G-3 with a rice aroma phenotype was obtained from an M2 population of 72-G-3.

### Example 3

A method for high-throughput targeted identification of an M1 generation plant mutant resulting from physical and chemical mutagenesis and for acquisition of the mutant as shown in FIG. 1 was provided, specifically including the following steps:

1. 100,000 seeds of the Oryza sativa variety Huazhan were irradiated with ⁶⁰Co-γ rays at a dose of 350 Gy to obtain an M1 generation of the Oryza sativa variety Huazhan.

2. Seedlings were cultivated using the 100,000 Huazhan M1 generation seeds; and then 96 plants, as a population, were planted in 8 rows, with 12 plants in each row. 500 populations, a total of 48,000 plants, were planted and numbered 1 to 500.

3. With each population as a unit, leaves were collected from each individual plant at an equal amount, and a total of 96 leaf samples were mixed in a centrifuge tube for DNA extraction. A total of 500 DNA samples were extracted and then numbered corresponding to the population numbers of 1 to 500.

4. With DNA of each population as a sequencing sample, targeted high-depth sequencing was conducted for transcription activator-like effector (TALE) binding regions of Xanthomonas oryzae in regulatory regions of the Os11N3 (Os11g0508600) and Os8N3 (Os08g0535200) genes at a sequencing depth of >5,000×.

5. The high-depth targeted sequencing data were aligned with a Nipponbare sequence to obtain the SNP and Indel data of the TALE binding regions of Xanthomonas oryzae in regulatory regions of the Os11N3 and Os8N3 genes in the 500 samples. According to test results, it was found that sample 261 had a 22 bp deletion of [CAACCAGGTGCTAAGCTCATC (SEQ ID NO: 29)/−] at 18174476-18174497 (RAP_Locus) of the Os11N3 gene; and according to test results, it was also found that sample 385 had a 34 bp deletion of [TCTCCCCCTACTGTACACCACCAAAAGTGGAGGG (SEQ ID NO: 30)/−] at 26728837-26728870 (RAP_Locus) of the Os8N3 gene.

6. The Indels in the sequencing data of the above samples 261 and 385 were verified by the dPCR technology. Verification results showed that the deletion mutation genotypes in the targeted sequencing data were present in the three samples, respectively.

7. According to the deletion mutation genotypes in the DNA of the population samples 261 and 385, the following KASP genotyping primers were designed:

261FAM 5′-GAAGGTGACCAAGTTCATGCTTCCTAGCACTATATAAACCCCCTC-3′ (as shown in SEQ ID NO: 13, in which an underlined part represents a fluorescent tag primer);

261HEX 5′-GAAGGTCGGAGTCAACGGATTTCCTAGCACTATATAAACCCCCTA-3′ (as shown in SEQ ID NO: 14, in which an underlined part represents a fluorescent tag primer);

261COMMON 5′-CTTGAGTTTGCTTTGCTTGAAGGC-3′ (as shown in SEQ ID NO: 15);

385FAM 5′-GAAGGTGACCAAGTTCATGCTGGCTCAGTGTTTATATAGTTGGAGAC-3′ (as shown in SEQ ID NO: 16, in which an underlined part represents a fluorescent tag primer);

385HEX 5′-GAAGGTCGGAGTCAACGGATTGGCTCAGTGTTTATATAGTTGGAGA-3′ (as shown in SEQ ID NO: 17, in which an underlined part represents a fluorescent tag primer); and

385COMMON 5′-GAAAAAAAAGCAAAGGTTAGATATGCA-3′ (as shown in SEQ ID NO: 18).

8. The KASP genotyping primers for the 2 deletion mutation loci were used to genotype the 96×2=192 individual plants in the populations 261 and 385. According to genotyping results, it was found that the 7th plant in row 2 of the population 261 (261-B-7) had a 22 bp deletion genotype of [CCAACCAGGTGCTAAGCTCATC (SEQ ID NO: 29)/−] in the TALE binding region of Xanthomonas oryzae in the regulatory region of the Os11N3 gene; and according to genotyping results, it was also found that the 3rd plant in row 8 of the population 385 (385-H-3) had a 34 bp deletion genotype of [TCTCCCCCTACTGTACACCACCAAAAGTGGAGGG (SEQ ID NO: 30)/−] in the TALE binding region of Xanthomonas oryzae in the regulatory region of the Os8N3 gene. Finally, 2 M1 generation individual plants with a deletion mutation of the target gene were obtained, including 1 chimera 261-B-7 with an Os11N3 gene frameshift mutation and 1 chimera 385-H-3 with an Os8N3 gene frameshift mutation.

9. DNA was extracted from leaves corresponding to each ear on each of the individual plants 261-B-7 and 385-H-3, and a sequencing test was conducted for the Indel mutation in each individual plant. It was found that 4 ears on the individual plant 261-B-7 had the mutation genotype of [CCAACCAGGTGCTAAGCTCATC (SEQ ID NO: 29)/−]; and 3 ears on the individual plant 385-H-3 had the mutation genotype of [TCTCCCCCTACTGTACACCACCAAAAGTGGAGGG (SEQ ID NO: 30)/−]. With each individual plant as a unit, seeds of ears with the mutation genotype were mixed-collected.

10. Seeds of each of 261-B-7 and 385-H-3 were grouped into 2 populations and then sown, and each individual plant was genotyped with the KASP primers designed in step 7. Finally, Xanthomonas oryzae-resistant Huazhan-261-B-7 and Huazhan-385-H-3 were obtained from M2 populations of 261-B-7 and 385-H-3, respectively.

### Example 4

The Oryza sativa OsNramp5⁻¹⁸ mutant gene obtained in Example 1 had a nucleotide sequence shown in SEQ ID NO: 19. According to the test results of Beijing Tsingke Biotechnology Co., Ltd., the Oryza sativa OsNramp5⁻¹⁸ mutant gene was obtained through an 18 bp deletion of [CCTACGTGGCAATTCACA (SEQ ID NO: 28)/−] at 8875646-8875663 (RAP_Locus) of the OsNramp5 gene, in which 1 bp was at a junction between intron 8 and exon 9.

The identification method of Example 1 shown in FIG. 1 could be used to determine whether a mutant sample resulting from physical and chemical mutagenesis includes the Oryza sativa OsNramp5⁻¹⁸ mutant gene.

Use of the Oryza sativa OsNramp5⁻¹⁸ mutant gene or the mutant carrying the Oryza sativa OsNramp5⁻¹⁸ mutant gene obtained in Example 1 in the selective breeding and preparation of an Oryza sativa variety with a low-cadmium-absorption phenotype was provided, specifically including the following steps:

1) the obtained mutant carrying the genetic phenotype of the OsNramp5⁻¹⁸ mutant gene was used as a donor parent and the Oryza sativa Huazhan was used as a recipient parent for crossbreeding to obtain F1 seeds;

2) the F1 seeds were sown, and then an individual plant was selected and crossed with the recurrent parent Oryza sativa Huazhan to obtain BC1F1 seeds;

3) the BC1F1 seeds were sown, each individual plant was subjected to BC1F1 population DNA extraction, the KASP genotyping primers designed in Example 1 were used to conduct background selection on individual plants carrying the Oryza sativa OsNramp5⁻¹⁸ mutant gene, and an individual plant with the highest background similarity to the recurrent parent was selected for backcrossing to obtain BC2F1 seeds;

4) the BC2F1 seeds were sown, each individual plant was subjected to BC2F1 population DNA extraction, the KASP genotyping primers designed in Example 1 were used to conduct background selection on individual plants carrying the Oryza sativa OsNramp5⁻¹⁸ mutant gene, and an individual plant with the highest background similarity to the recurrent parent was selected for backcrossing to obtain BC3F1 seeds;

5) the BC3F1 seeds were sown, each individual plant was subjected to BC3F1 population DNA extraction, the KASP genotyping primers designed in Example 1 were used to conduct background selection on individual plants carrying the Oryza sativa OsNramp5⁻¹⁸ mutant gene, and an individual plant with highest background similarity to the recurrent parent was selected for selfing to obtain BC3F2 seeds; and

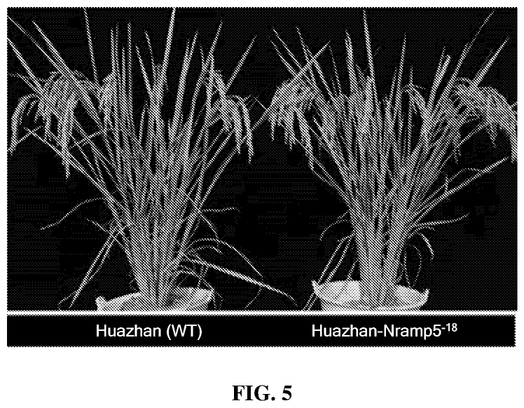

6) the BC3F2 seeds were sown, each individual plant was subjected to BC3F2 population DNA extraction, the KASP genotyping primers designed in Example 1 were used to conduct background selection on individual plants carrying the Oryza sativa OsNramp5⁻¹⁸ mutant gene, and an individual plant Huazhan-Nramp5⁻¹⁸ with the same genetic background as the Oryza sativa Huazhan was selected and harvested, which was the Oryza sativa variety Huazhan-Nramp5⁻¹⁸ with a low-cadmium-absorption phenotype shown in FIG. 5.

The Oryza sativa variety Huazhan-Nramp5⁻¹⁸ with a low-cadmium-absorption phenotype obtained above was subjected to a cadmium stress potting test. A soil was taken from a surface layer of a field, and the basic fertility thereof was tested. The soil was air-dried, sieved, subjected to impurity removal, thoroughly mixed, and dispensed into each pot at 25 mg/kg, then a Cd(CdCl₂) solution was added to the standby soil at a cadmium concentration of 2 mg/kg, and then the soil was equilibrated for 4 weeks.

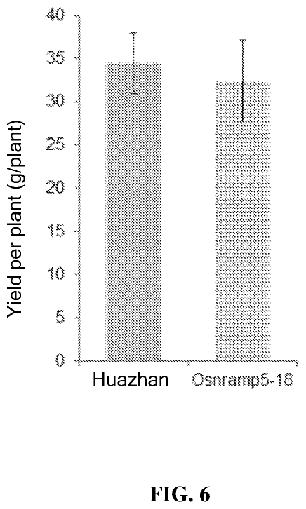

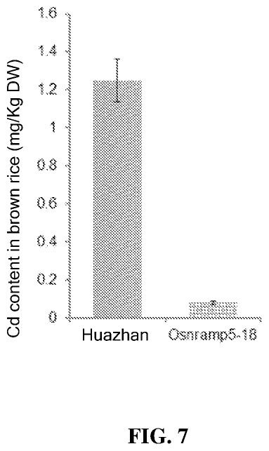

The mutant Huazhan-Nramp5⁻¹⁸ obtained in step 6) of this example and a Huazhan control were planted in the cadmium-contaminated pots, with 6 holes per pot and 2 plants per hole. 3 replicates were set for each test sample. Samples were taken at a maturity stage of Oryza sativa. Stems, leaves, and ears of an individual plant were separated, the ears were subjected to seed-husking, and the stems and leaves were rinsed with tap water and then washed with deionized water. Each sample was deactivated at 105° C. for 30 min and then dried at 80° C. to a constant weight. A dry weight of seeds per plant was determined, and results were shown in FIG. 6. The brown rice was crushed, sieved through a 100-mesh sieve, and digested with HNO₃—HClO₄, then a cadmium content and contents of other related mineral elements in the brown rice were determined by an inductively coupled plasma-atomic emission spectrometer (ICP-AES), and results were shown in FIG. 7. The results in FIG. 6 and FIG. 7 show that a Cd content in the brown rice of the mutant is significantly lower than that of the control, while a biological yield of the mutant is not significantly different from that of the control.

### Example 5

The Oryza sativa OsRR22⁻¹ mutant gene obtained in Example 1 had a nucleotide sequence shown in SEQ ID NO: 20. According to the test results of Beijing Tsingke Biotechnology Co., Ltd., the Oryza sativa OsRR22⁻¹ mutant gene was obtained through a 1 bp deletion of [G/−] (at 4138902) in exon 3 of the Oryza sativa OsRR22 gene.

The identification method of Example 1 shown in FIG. 1 could be used to determine whether a mutant sample resulting from physical and chemical mutagenesis includes the Oryza sativa OsRR22⁻¹ mutant gene.

Use of the Oryza sativa OsRR22⁻¹ mutant gene or the mutant carrying the Oryza sativa OsRR22⁻¹ mutant gene obtained in Example 1 in the selective breeding and preparation of an Oryza sativa variety with a low-cadmium-absorption phenotype was provided, specifically including the following steps:

1) the mutant carrying the genetic phenotype of the OsRR22⁻¹ mutant gene obtained in Example 1 was used as a donor parent and the Oryza sativa Huazhan was used as a recipient parent for crossbreeding to obtain F1 seeds;

2) the F1 seeds were sown, and then an individual plant was selected and crossed with the recurrent parent Oryza sativa Huazhan to obtain BC1F1 seeds;

3) the BC1F1 seeds were sown, each individual plant was subjected to BC1F1 population DNA extraction, the KASP genotyping primers designed in Example 1 were used to conduct background selection on individual plants carrying the Oryza sativa OsRR22⁻¹ mutant gene, and an individual plant with the highest background similarity to the recurrent parent was selected for backcrossing to obtain BC2F1 seeds;

4) the BC2F1 seeds were sown, each individual plant was subjected to BC2F1 population DNA extraction, the KASP genotyping primers designed in Example 1 were used to conduct background selection on individual plants carrying the Oryza sativa OsRR22⁻¹ mutant gene, and an individual plant with the highest background similarity to the recurrent parent was selected for backcrossing to obtain BC3F1 seeds;

5) the BC3F1 seeds were sown, each individual plant was subjected to BC3F1 population DNA extraction, the KASP genotyping primers designed in Example 1 were used to conduct background selection on individual plants carrying the Oryza sativa OsRR22⁻¹ mutant gene, and an individual plant with highest background similarity to the recurrent parent was selected for selfing to obtain BC3F2 seeds; and

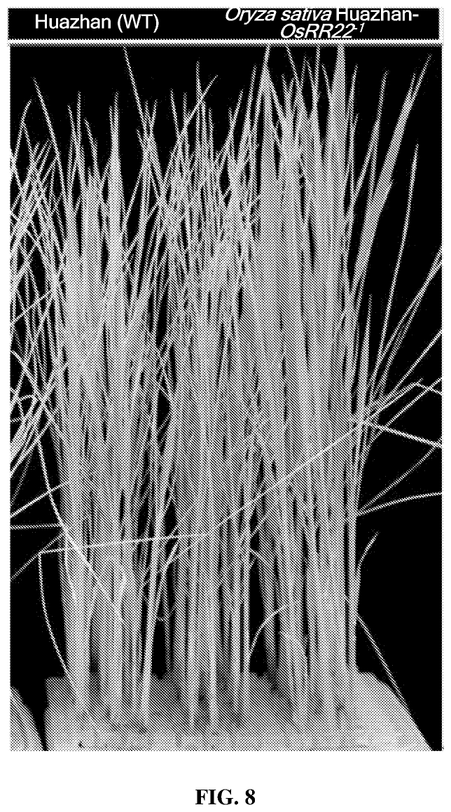

6) the BC3F2 seeds were sown, each individual plant was subjected to BC3F2 population DNA extraction, the KASP genotyping primers designed in Example 1 were used to conduct background selection on individual plants carrying the Oryza sativa OsRR22⁻¹ mutant gene, and an individual plant Huazhan-OsRR22⁻¹ with the same genetic background as the Oryza sativa Huazhan was selected and harvested, which was the Oryza sativa variety Huazhan-OsRR22⁻¹ with a salt-tolerant phenotype shown in FIG. 8.

Seedlings were simultaneously cultivated for the mutant Huazhan-OsRR22⁻¹ obtained in this example and Huazhan (WT). At the three-complete leaf and one-spear leaf stage, Oryza sativa seedlings were placed in a 0.8% sodium chloride aqueous solution to undergo a continuous stress treatment for 10 d, and then photographed. 10 plants were collected from each of the mutant and control groups and rinsed with pure water, then the residual water was removed, and a root length, a plant height, and a fresh seedling weight were measured. The measurement results were shown in Table 1 below.

TABLE 1
Comparison results of salt tolerance between
Huazhan-OsRR22⁻¹ and Huazhan (WT)

| | Root length | Plant height | Fresh seedling |
|---|---|---|---|
| Sample | (mm) | (mm) | weight (g) |
| Huazhan (WT) | 89.37 ± 2.31 | 109.10 ± 1.42 | 0.17 ± 0.01 |
| Huazhan-OsRR22⁻¹ | 108 ± 4.12 | 118 ± 4.22 | 0.25 ± 0.01 |

It can be seen from the results in Table 1 that the Huazhan (WT) withered after 10 d of the stress treatment, while the mutant Huazhan-OsRR22⁻¹ still grew normally; and the root length, plant height, and fresh seedling weight of the mutant were also significantly higher than that of the Huazhan (WT), indicating that the growth potential of the mutant Huazhan-OsRR22⁻¹ under salt stress was significantly stronger than that of the WT control.

### Example 6

The Oryza sativa OsRR22⁻⁷ mutant gene obtained in Example 1 had a nucleotide sequence shown in SEQ ID NO: 21. According to the test results of Beijing Tsingke Biotechnology Co., Ltd., the Oryza sativa OsRR22⁻⁷ mutant gene was obtained through a 7 bp deletion of KGGCTTT/−1 (at 4140861-4140867) in exon 5 of the Oryza sativa OsRR22 gene.

The identification method of Example 1 shown in FIG. 1 could be used to determine whether a mutant sample resulting from physical and chemical mutagenesis includes the Oryza sativa OsRR22⁻⁷ mutant gene.

Use of the Oryza sativa OsRR22⁻⁷ mutant gene or the mutant carrying the Oryza sativa OsRR22⁻⁷ mutant gene obtained in Example 1 in the selective breeding and preparation of an Oryza sativa variety with a low-cadmium-absorption phenotype was provided, specifically including the following steps:

1) the mutant carrying the genetic phenotype of the OsRR22⁻⁷ mutant gene obtained in Example 1 was used as a donor parent and the Oryza sativa Huazhan was used as a recipient parent for crossbreeding to obtain F1 seeds;

2) the F1 seeds were sown, and then an individual plant was selected and crossed with the recurrent parent Oryza sativa Huazhan to obtain BC1F1 seeds;

3) the BC1F1 seeds were sown, each individual plant was subjected to BC1F1 population DNA extraction, the KASP genotyping primers designed in Example 1 were used to conduct background selection on individual plants carrying the Oryza sativa OsRR22⁻⁷ mutant gene, and an individual plant with the highest background similarity to the recurrent parent was selected for backcrossing to obtain BC2F1 seeds;

4) the BC2F1 seeds were sown, each individual plant was subjected to BC2F1 population DNA extraction, the KASP genotyping primers designed in Example 1 were used to conduct background selection on individual plants carrying the Oryza sativa OsRR22⁻⁷ mutant gene, and an individual plant with the highest background similarity to the recurrent parent was selected for backcrossing to obtain BC3F1 seeds;

5) the BC3F1 seeds were sown, each individual plant was subjected to BC3F1 population DNA extraction, the KASP genotyping primers designed in Example 1 were used to conduct background selection on individual plants carrying the Oryza sativa OsRR22⁻⁷ mutant gene, and an individual plant with highest background similarity to the recurrent parent was selected for selfing to obtain BC3F2 seeds; and

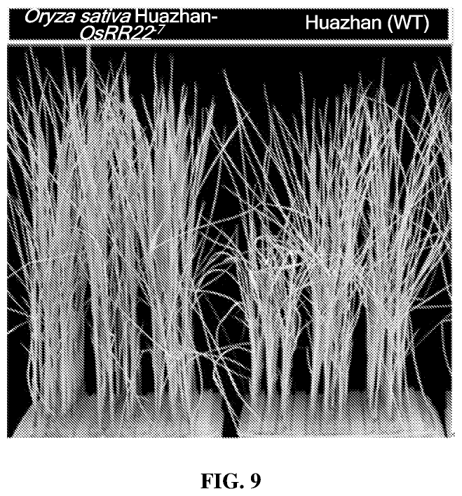

6) the BC3F2 seeds were sown, each individual plant was subjected to BC3F2 population DNA extraction, the KASP genotyping primers designed in Example 1 were used to conduct background selection on individual plants carrying the Oryza sativa OsRR22⁻⁷ mutant gene, and an individual plant Huazhan-OsRR22⁻⁷ with the same genetic background as the Oryza sativa Huazhan was selected and harvested, which was the Oryza sativa variety Huazhan-OsRR22⁻⁷ with a salt-tolerant phenotype shown in FIG. 9.

Seedlings were simultaneously cultivated for the mutant Huazhan-OsRR22⁻⁷ obtained in this example and Huazhan (WT). At the three-complete leaf and one-spear leaf stage, Oryza sativa seedlings were placed in a 0.8% sodium chloride aqueous solution to undergo a continuous stress treatment for 10 d, and then photographed. 10 plants were collected from each of the mutant and control groups and rinsed with pure water, then the residual water was removed, and a root length, a plant height, and a fresh seedling weight were measured. The measurement results were shown in Table 2 below.

TABLE 2
Comparison results of salt tolerance between
Huazhan-OsRR22⁻⁷ and Huazhan (WT)

| | Root length | Plant height | Fresh seedling |
|---|---|---|---|
| Sample | (mm) | (mm) | weight (g) |
| Huazhan (WT) | 87.37 ± 2.24 | 106.10 ± 1.22 | 0.16 ± 0.01 |
| Huazhan-OsRR22-7 | 107 ± 3.12 | 116 ± 4.34 | 0.23 ± 0.01 |

It can be seen from the results in Table 2 that the Huazhan (WT) withered after 10 d of the stress treatment, while the mutant Huazhan-OsRR22⁻⁷ still grew normally; and the root length, plant height, and fresh seedling weight of the mutant were also significantly higher than that of the Huazhan (WT), indicating that the growth potential of the mutant Huazhan-OsRR22⁻⁷ under salt stress was significantly stronger than that of the WT control.

### Example 7

A method for high-throughput targeted identification of an M1 generation Sorghum bicolor mutant resulting from physical and chemical mutagenesis and for acquisition of the mutant as shown in FIG. 1 was provided, specifically including the following steps:

1. 20,000 seeds of the Sorghum bicolor variety 654 were taken and soaked in water for 16 h in a constant-temperature incubator at 28° C., and then taken out and air-dried; the seeds were then soaked in a 1% (w/w) EMS solution for 8 h in a constant-temperature incubator at 28° C., and then taken out and air-dried; and the seeds were rinsed with water 8 times, subjected to germination at 37° C., and sown for seedling cultivation, and resulting seedlings were planted. 96 plants, as a population, were planted in 8 rows, with 12 plants in each row. 100 populations, a total of 9,600 plants, were planted and numbered 1 to 100.

2. With each population as a unit, leaves were collected from each individual plant at an equal amount, and a total of 96 leaf samples were mixed in a centrifuge tube for DNA extraction. A total of 100 DNA samples were extracted and then numbered corresponding to the population numbers of 1 to 100.

3. With DNA of each population as a sequencing sample, targeted high-depth sequencing was conducted for an exon region of the ALS (GenBank: LN898467.1) gene at a sequencing depth of >5,000×.

4. The high-depth targeted sequencing data were aligned with the reference genome Sorghum_bicolor NCBIv3 to obtain the SNP and Indel data of the exon of the ALS gene in the 100 samples. According to test results, it was found that sample 68 had a single base mutation of [G/A] at base 1,871 of the only exon of the ALS gene, which caused an amino acid 624 to change from Ser to Asn.

5. The SNP in the sequencing data of the above population sample 68 was verified by the dPCR technology. Verification results showed that the SNP genotype in the targeted sequencing data was present in this sample.

6. According to the SNP genotype in the DNA of the population sample 68, the following KASP genotyping primers were designed:

68FAM
(SEQ ID NO: 22)
5’-GAAGGTGACCAAGTTCATGCTAGCATGTGTTGCCTATGATCCCTA
G-3’
68HEX
(SEQ ID NO: 23)
5’-GAAGGTCGGAGTCAACGGATTAGCATGTGTTGCCTATGATCCCTA
A-3’
68COMMON
(SEQ ID NO: 24)
5’-TCAATACACAGTCCTGCCATCACC-3’.

7. The KASP genotyping primers designed above were used to genotype the 96 individual plants in the population 68. According to genotyping results, it was found that the 8th plant in row 6 of the population 68 (68-F-8) had the single base mutation genotype of [G/A] at base 1,871 of the ALS gene. Finally, an individual plant in which the amino acid 624 in an ALS protein encoded by the target gene changed from Ser to Asn was obtained.

8. DNA was extracted from leaves corresponding to each ear on the individual plant 68-F-8, and a sequencing test was conducted for the SNP mutation in the individual plant. It was found that 5 ears on the individual plant 68-F-8 had the single base mutation genotype of [G/A]. Seeds of ears with the mutation genotype were mixed-collected.

9. Seedlings were cultivated using the seeds of the individual plant 68-F-8, then each individual seedling was planted independently, and each individual plant was genotyped with the KASP primers designed in step 6 above. Finally, Sorghum bicolor with an imidazolinone herbicide-resistant phenotype was obtained from an M2 population of 68-F-8.

### Example 8

A method for high-throughput targeted identification of an M1 generation Zea mays mutant resulting from physical and chemical mutagenesis and for acquisition of the mutant as shown in FIG. 1 was provided, specifically including the following steps:

1. 20,000 seeds of the Zea mays variety 345 (hereinafter referred to as 345) were irradiated with 80 MeV/u carbon ions (¹²C⁶⁺) at a dose of 180 Gy to obtain an M1 generation of the Zea mays variety 345.

2. Seedlings were cultivated using the 20,000 345 M1 generation seeds, and then each individual seedling was planted independently. 96 plants, as a population, were planted in 8 rows, with 12 plants in each row. 100 populations, a total of 9,600 plants, were planted and numbered 1 to 100.

3. With each population as a unit, leaves were collected from each individual plant at an equal amount, and a total of 96 leaf samples were mixed in a centrifuge tube for DNA extraction. A total of 100 DNA samples were extracted and then numbered corresponding to the population numbers of 1 to 100.

4. With DNA of each population as a sequencing sample, targeted high-depth sequencing was conducted for an exon region of the ZmTMS5 gene at a sequencing depth of >5,000×.

5. The high-depth targeted sequencing data were aligned with a Zea mays reference genome B73_RefGen_v4 to obtain the SNP and Indel data of the exon of the ZmTMS5 gene in the 100 samples. According to test results, it was found that sample 54 had a 1 bp deletion of [C/−] at a base 38 of exon 1 of the ZmTMS5 gene.

6. The Indel in the sequencing data of the population sample 54 was verified by the dPCR technology. Verification results showed that the deletion mutation genotype in the targeted sequencing data was present in this sample.

7. According to the deletion mutation genotype in the DNA of the population sample 54, the following KASP genotyping primers were designed:

38FAM
(SEQ ID NO: 25)
5’-GAAGGTGACCAAGTTCATGCTCGAGTCCACTGTGGAGGTTCC-3’
38HEX
(SEQ ID NO: 26)
5’-GAAGGTCGGAGTCAACGGATTCGAGTCCACTGTGGAGGTTCT-3’
and
38COMMON
(SEQ ID NO: 27)
5’-ACCCCTCGATCTCTATACGGTG-3’

The KASP genotyping primers designed above were used to genotype the 96 individual plants in the population 54. According to genotyping results, it was found that the 3rd plant in row 4 of the population 54 (54-D-3) had the single base mutation genotype of [C/−] at base 38 of the ZmTMS5 gene.

8. DNA was extracted from leaves corresponding to each ear on the individual plant 54-D-3, and a sequencing test was conducted for the Indel mutation in the individual plant. It was found that 4 ears on the individual plant 54-D-3 had the single base deletion mutation genotype of [C/−]. Seeds of ears with the mutation genotype were mixed-collected.

9. Seedlings were cultivated using the seeds of the individual plant 54-D-3, then each individual seedling was planted independently, and each individual plant was genotyped with the KASP primers designed in step 6 above. Finally, a Zea mays variety 345 with thermo-sensitive male sterility was obtained from an M2 population of 54-D-3.

What is claimed is:

1. A method for high-throughput targeted identification of an M1 generation plant mutant resulting from physical and chemical mutagenesis, comprising the following steps:
a) mutagenizing plants by the physical and chemical mutagenesis at a non-lethal dose to obtain an M1 generation plant material;
b) planting each individual plant in the M1 generation plant material independently, collecting leaves from each planted individual plant, and mixing leaves collected from an individual plant;
c) extracting a mixed pool of DNA from a mixed leaf material;
d) subjecting the mixed pool of DNA to high-depth targeted sequencing for a target gene region; and
e) aligning a high-depth targeted sequencing result with a related sequence of the target gene region to identify whether a population DNA sample in the high-depth targeted sequencing result comprises target single nucleotide polymorphisms (SNPs) and/or Indels of the target gene region.

2. The method according to claim 1, wherein after the identification in step e) is completed, the method further comprises using one or more verification modes to verify an identification result, such as to determine whether the sample comprises the target SNPs and/or Indels of the target gene region; and the verification mode specifically comprises:
e₁: testing all plants by a digital polymerase chain reaction (dPCR) identification mode for verification;
e₂: genotyping each individual plant by a Kompetitive Allele Specific PCR (KASP) genotyping mode for verification; and
e₃: verifying each individual plant by a Sanger sequencing mode.

3. The method according to claim 2, wherein the verification mode comprises the following two steps:
1) detecting population DNA in the high-depth targeted sequencing result by the dPCR identification mode to identify whether the population DNA sample comprises target SNPs and/or Indels of the target gene region, and if so, proceeding to step 2), otherwise, finishing; and
2) based on SNP and/or Indel loci identified by dPCR, designing a KASP genotyping primer, and subjecting each individual plant in a population corresponding to a mixed pool sample of the plant mutant to KASP genotyping to finally determine whether there is a chimeric individual plant with a mutation in the target gene region.

4. The method according to claim 3, wherein the related sequence of the target gene region is a gene sequence of Oryza sativa OsNramp5 gene, there is a 18 bp deletion of [CTACGTGGCAATTCACA (SEQ ID NO: 28)/−] at 8875646-8875663 from a junction between intron 8 and exon 9 of the OsNramp5 gene, and the KASP genotyping primer comprises the following sequences:
FAM
(SEQ ID NO: 1)
5’-GAAGGTGACCAAGTTCATGCTGAAGAACCTGCACCCGTCCT-3’,
HEX
(SEQ ID NO: 2)
5-’GAAGGTCGGAGTCAACGGATTGAAGAACCTGCACCCGTCAC-’3,
and
COMMON
(SEQ ID NO: 3)
5’-GCATGGAAAGAAACTGAACAAAGAT-3’.

5. The method according to claim 3, wherein the related sequence of the target gene region is a gene sequence of Oryza sativa OsRR22 gene, there is a 1 bp deletion of [G/−] at 4138902 in exon 3 of the OsRR22 gene, and the KASP genotyping primer comprises the following sequences:
FAM
(SEQ ID NO: 4)
5’-GAAGGTGACCAAGTTCATGCTCAGGCACCATGAGTTATCCCT-3’,
HEX
(SEQ ID NO: 5)
5’-GAAGGTCGGAGTCAACGGATTCAGGCACCATGAGTTATCCCC-3’,
and
COMMON
(SEQ ID NO: 6)
5’-’TGTTATCAGTAAATGGAGAGACAAAGAC-3’.

6. The method according to claim 3, wherein the related sequence of the target gene region is a gene sequence of Oryza sativa OsRR22 gene, there is a 7 bp deletion of [CGGCTTT/−] at 4140861-4140867 in exon 5 of the OsRR22 gene, and the KASP genotyping primer comprises the following sequences:
FAM
(SEQ ID NO: 7)
5-’GAAGGTGACCAAGTTCATGCTGCAAGCTCCTGAAGTCCGAA-’3
(SEQ ID NO: 7),
HEX
(SEQ ID NO: 8)
5’-GAAGGTCGGAGTCAACGGATTCAAGCTCCTGAAGTCCGCG-3’,
and
COMMON
(SEQ ID NO: 9)
5’-TTCTGCTGCTCTTCCATCTTTCA-3’.

7. The method according to claim 1, wherein the non-lethal dose in step a) refers to controlling a dose within a range of 20% higher and lower a median-lethal dose.

8. The method according to claim 1, wherein the physical and chemical mutagenesis in step a) comprises one or two selected from the group consisting of physical mutagenesis and chemical mutagenesis;
the physical mutagenesis comprises ultraviolet (UV) mutagenesis, X-ray mutagenesis, γ-ray mutagenesis, β-ray mutagenesis, α-ray mutagenesis, high-energy particle mutagenesis, cosmic ray mutagenesis, and microgravity mutagenesis;
the chemical mutagenesis comprises alkylating agent mutagenesis, azide mutagenesis, base analog mutagenesis, lithium chloride mutagenesis, antibiotic mutagenesis, and intercalative dye mutagenesis; and
the alkylating agent mutagenesis comprises ethyl methanesulfonate (EMS) mutagenesis, diethyl sulfate (DES) mutagenesis, and ethyleneimine (EI) mutagenesis.

9. The method according to claim 1, wherein in step b), when each individual plant in the M1 generation plant material is planted independently, an arbitrary number of plants are clustered as a population, and each population is numbered; and in step c), leaves of each population are mixed in a centrifuge tube for DNA extraction.

10. The method according to claim 9, wherein the population comprises 48, 96, or 192 plants; during the leaf collection, leaves are collected from different parts of a same individual plant at a same amount; and
in step d), a sequencing depth of the high-depth targeted sequencing for a single population with 48 plants is greater than 2,000×, a sequencing depth of the high-depth targeted sequencing for a single population with 96 plants is greater than 5,000×, and a sequencing depth of the high-depth targeted sequencing for a single population with 192 plants is greater than 10,000×.

11. The method according claim 1, wherein in step d), the target gene region comprises an exon region of a target gene or a non-coding region of the target gene; the high-depth targeted sequencing comprises multiplex PCR-based targeted capture technology, liquid-phase probe hybridization capture-based targeted capture technology, or third-generation sequencing-based single-molecule targeted sequencing technology; and
a sequencing depth of the high-depth targeted sequencing is determined according to a number of individual plants in each population.

12. The method according to claim 1, wherein the target gene is the Oryza sativa OsNramp5 gene or the Oryza sativa OsRR22 gene.

13. The method according to claim 1, wherein on the basis of identifying a chimeric individual plant with a mutation of a target gene region by the method, the method further comprises the following steps to obtain a mutant:
f) for each chimeric individual plant with the mutation of the target gene region, extracting DNA of leaves corresponding to each ear for DNA identification, selecting an ear with the mutation, and mixed-collecting seeds; and
g) mixed-sowing seeds obtained from the mixed-collecting, and collecting leaves from each individual plant independently for DNA identification to finally obtain an M2 individual plant with a target genetic phenotype.

14. The method according to claim 13, wherein the chimeric individual plant with the mutation of the target gene region refers to a chimeric individual plant with the Oryza sativa OsNramp5⁻¹⁸ mutant gene, the Oryza sativa OsRR22⁻¹ mutant gene, or the Oryza sativa OsRR22⁻⁷ mutant gene;
compared with the OsNramp5 gene sequence of Nipponbare, the Oryza sativa OsNramp5⁻¹⁸ mutant gene comprises a 18 bp deletion of [CTACGTGGCAATTCACA (SEQ ID NO: 28)/−] at 8875646-8875663 (RAP_Locus) in exon 9;
compared with the OsRR22 gene sequence of Nipponbare, the Oryza sativa OsRR22⁻¹ mutant gene comprises a 1 bp deletion of [G/−] at 4138902 (RAP_Locus) in exon 3 of the OsRR22 gene; and
compared with the OsRR22 gene sequence of Nipponbare, the Oryza sativa OsRR22⁻⁷ mutant gene comprises a 7 bp deletion of [CGGCTTT/−] at 4140861-4140867 (RAP_Locus) in exon 5.

15. The method according to claim 13, wherein in step f), the DNA identification refers to identification with a designed KASP genotyping primer; and
in step g), the DNA identification refers to identification with a designed KASP genotyping primer.

16. An Oryza sativa mutant gene obtained in the identification of an M1 generation Oryza sativa mutant resulting from physical and chemical mutagenesis by the method according to claim 12, wherein the Oryza sativa mutant gene is an Oryza sativa OsNramp5⁻¹⁸ mutant gene, an Oryza sativa OsRR22⁻¹ mutant gene, or an Oryza sativa OsRR22⁻⁷ mutant gene;
compared with the OsNramp5 gene sequence of Nipponbare, the Oryza sativa OsNramp5⁻¹⁸ mutant gene comprises a 18 bp deletion of [CCTACGTGGCAATTCACA (SEQ ID NO: 28)/−] at 8875646-8875663 (RAP_Locus) in exon 9;
compared with theOsRR22 gene sequence of Nipponbare, the Oryza sativa OsRR22⁻¹ mutant gene comprises a 1 bp deletion of [G/−] at 4138902 (RAP_Locus) in exon 3 of the OsRR22 gene; and
compared with the OsRR22 gene sequence of Nipponbare, the Oryza sativa OsRR22⁻⁷ mutant gene comprises a 7 bp deletion of [CGGCTTT/−] at 4140861-4140867 (RAP_Locus) in exon 5.

17. The Oryza sativa mutant gene according to claim 16, wherein the Oryza sativa OsNramp5⁻¹⁸ mutant gene has a nucleotide sequence shown in SEQ ID NO: 19, or a truncated sequence of the nucleotide sequence, or a specific sequence that has 95% or more homology with the nucleotide sequence and encodes the same functional protein as the nucleotide sequence;
the Oryza sativa OsRR22⁻¹ mutant gene has a nucleotide sequence shown in SEQ ID NO: 20, or a truncated sequence of the nucleotide sequence, or a specific sequence that has 95% or more homology with the nucleotide sequence and encodes the same functional protein as the nucleotide sequence; and
the Oryza sativa OsRR22⁻⁷ mutant gene has a nucleotide sequence shown in SEQ ID NO: 21, or a truncated sequence of the nucleotide sequence, or a specific sequence that has 95% or more homology with the nucleotide sequence and encodes the same functional protein as the nucleotide sequence.

18. Use of the Oryza sativa mutant gene according to claim 16 in molecular marker-assisted breeding (MAB) of a crop.

19. The use according to claim 18, wherein
when the Oryza sativa mutant gene is the Oryza sativa OsNramp5⁻¹⁸ mutant gene, the Oryza sativa OsNramp5⁻¹⁸ mutant gene or a mutant carrying the Oryza sativa OsNramp5⁻¹⁸ mutant gene is used in the selective breeding or preparation of an Oryza sativa variety with a low-cadmium-absorption phenotype; and
when the Oryza sativa mutant gene is the Oryza sativa OsRR22⁻¹ mutant gene or the Oryza sativa OsRR22⁻⁷ mutant gene, the Oryza sativa OsRR22⁻¹ or OsRR22⁻⁷ mutant gene or a mutant carrying the Oryza sativa OsRR22⁻¹ or OsRR22⁻⁷ mutant gene is used in the selective breeding or preparation of an Oryza sativa variety with a salt-tolerant phenotype.
